(19) United States
(12) Patent Application Publication
(10) Pub. No.: US 2024/0023896 A1
(43) Pub. Date: Jan. 25, 2024

(54) STENT DELIVERY SYSTEM AND STENT SENSOR DEVICE

(71) Applicant: Terumo Kabushiki Kaisha, Tokyo (JP)

(72) Inventor: Kaori ENOMOTO, Kanagawa (JP)

(73) Assignee: Terumo Kabushiki Kaisha, Tokyo (JP)

(21) Appl. No.: 18/354,693

(22) Filed: Jul. 19, 2023

(30) Foreign Application Priority Data
Jul. 21, 2022 (JP) 2022-116740

(51) Int. Cl. A61B 5/00; A61F 2/966; A61B 5/026; A61B 5/0215

(52) U.S. Cl. CPC A61B 5/6862; A61F 2/966; A61B 5/026; A61B 5/0215; A61B 5/0031; A61F 2250/0096; A61F 2002/823

(57) ABSTRACT

A stent delivery system that obtains biological-related information or stent state information in a stent using a signal from a sensor after the stent indwells in a living body and then removing the sensor from the living body. A stent delivery system includes a sheath in which a stent is accommodated, a stent extrusion member, and a sensor device. The sensor device includes a sensor unit and a pressing portion configured to bring the sensor unit into contact with an inner surface of the stent indwelling into the living body. The sensor device can move the sensor unit in a rear end direction of the stent by movement of the stent extrusion member toward a rear end side or withdrawal of the sensor device. The sensor device can be taken out from the living body by removing the stent delivery system or the sensor device from the living body.

### CROSS-REFERENCES TO RELATED APPLICATIONS

This application is based on and claims priority to Japanese Patent Application No. 2022-116740 filed on Jul. 21, 2022, the entire content of which is incorporated herein by reference.

### TECHNOLOGICAL FIELD

The present invention generally relates to a stent delivery system that is used to ameliorate a stenosed site or an occlusion site generated in a body lumen such as a blood vessel (for example, a cerebral blood vessel or a coronary artery of a heart), a bile duct, a trachea, an esophagus, or a urethra, and more particularly to a stent delivery system and a stent sensor device that can obtain biological-related information or stent state information on a stent indwelling site.

### BACKGROUND DISCUSSION

An in-vivo indwelling stent is used to treat various diseases caused by stenosis or occlusion of a blood vessel or other body lumens. The stent is formed in a tubular shape in order to expand the stenosed site or the occlusion site and to secure the lumen thereof.

Since the stent is inserted into the body from outside the body, the stent has a compressed small diameter at the time of insertion, is expanded to increase a diameter at a target stenosed or occlusion site, and holds the lumen in the open state.

As the stent, a cylindrical stent obtained by processing a metal wire material or a metal tube is generally used. The stent is mounted on a catheter or the like in a compressed state, inserted into the living body, expanded in some way at the target site, and pressed against and fixed to an inner wall of the lumen to maintain a shape of the lumen.

Stents are classified into self-expandable stents and balloon inflatable stents according to a function and an indwelling method. The balloon inflatable stent does not have an expansion function by itself. After the stent mounted on a balloon is inserted into the target site, the balloon is inflated, and the stent is expanded (plastically deformed) by an inflation force of the balloon and is pressed and fixed to an inner surface of a target lumen. This type of stent requires a stent expansion operation as described above. On the other hand, the self-expandable stent has an expansion function by itself. The self-expandable stent is inserted into the living body in a thin and compressed state and is released from its compressed state at the target site, so that the self-expandable stent returns to an original expanded state thereof and is pressed to and fixed to the inner wall of the lumen to maintain the shape of the lumen.

One of causes of ischemic cerebral artery disease is stenosis and occlusion of intracranial artery and an aneurysm. As a general treatment method, a risk can be reduced by an anti-platelet therapy. However, there is a limit to a medical treatment, and a treatment by balloon inflation or stent indwelling is performed on a patient who exhibits drug resistance.

U.S. Pat. No. 9,439,791 discloses a stent for treating a body-cavity such as an embolization of a vascular aneurysm and the like. The stent disclosed in this patent is a so-called self-expandable stent, and is a stent having a generally cylindrical body formed of a single woven nitinol wire. A distal end portion and a proximal end portion of the stent include a plurality of loops, some of which include marker members used for visualizing a position of the stent, and further include an inner flow diverting layer.

U.S. Pat. No. 8,187,317 discloses an endoprosthesis (stent). The endoprosthesis disclosed in this patent is designed to be implanted into an aneurysm (12) of a patient caused by deformation of a vascular wall, strictly speaking, a wall of an artery (14). The endoprosthesis includes tubular envelopes (22, 24) extending in a direction (Y-Y′) and a plurality of pressure probes (26) fixed to the envelope (22, 24). Each probe (26) includes a sensor (28) that measures a pressure, and units (34, 36) that transmit a measurement value of the pressure to a monitoring device disposed outside a body of the patient. The units (34, 36) that transmit the measurement value of the pressure from each probe (26) are designed to generate an electromagnetic pressure measurement transmission signal having at least one distinguishable characteristic of the probe (26) of which these units (34, 36) form a part thereof.

However, although the stent disclosed in U.S. Pat. No. 9,439,791 is effective as a stent, it is not possible to obtain biological-related information or stent state information in the stent indwelling into the living body.

In U.S. Pat. No. 8,187,317, the endoprosthesis (stent) includes the pressure probes (26), and each probe (26) of the endoprosthesis (stent) can measure the pressure in the stent indwelling into the living body. However, since the pressure probe (26) is fixed to the endoprosthesis (stent), physical properties of the endoprosthesis (stent) in a pressure probe portion are different from those in other portions. For this reason, in this portion, favorable deformation of the endoprosthesis (stent) may be inhibited, and a thrombus may be formed at the endoprosthesis.

The stent delivery system and stent sensor device disclosed here are capable of obtaining biological-related information or stent state information in a stent after the stent indwells in a living body, and that are capable of taking out, from the living body, a sensor that collects the biological-related information or the stent state information.

### SUMMARY

(1) A stent delivery system includes: a stent that has a plurality of side wall openings, that is formed in a substantially cylindrical shape and compressed in a central axis direction for insertion into a living body, and that is expandable outward for implanting into the living body; a sheath possessing a distal portion accommodates the stent; and a stent extrusion member whose distal side portion is located in the distal portion of the sheath. The stent delivery system is configured to release the stent by moving the sheath toward a proximal end side with respect to the stent extrusion member. The stent delivery system further includes a sensor device, wherein the sensor device includes: a sensor unit; and a pressing portion capable of bringing the sensor unit into contact with an inner surface of the stent indwelling into the living body. The sensor device is able to the sensor unit in a proximal end direction of the stent by movement of the stent extrusion member toward the proximal end side or withdrawal of the sensor device, and the stent delivery system is able to obtain biological-related information or stent state information in the stent using a signal from the sensor unit. The sensor device is removable from the living body by removing the stent delivery system from the living body or removing the sensor device from the stent delivery system.

(2) The stent delivery system according to (1), the sensor device possesses two or more of the sensor units that are separated from each other by a predetermined distance for detecting at a plurality of positions in an axial direction of the stent.

(3) The stent delivery system according to (1) or (2), in which the pressing portion possesses a wire-shaped portion and the sensor unit located at a distal portion of the wire-shaped portion.

(4) The stent delivery system according to (3), in which the wire-shaped portion is formed a coil shape.

(5) The stent delivery system according to any one of (3), in which the wire-shaped portion possesses a plurality of helical shape wires.

(6) The stent delivery system according to any one of (1) to (5), in which the pressing portion is a wire-shaped pressing portion, and a rear end portion of the pressing portion is fixed to the stent extrusion member.

(7) The stent delivery system according to any one of (1) to (6), in which the pressing portion is a wire-shaped pressing portion, the sensor device comprises a traction wire, and a rear end portion of the pressing portion is interlocked with the traction wire.

(8) The stent delivery system according to any one of (1) to (7), in which the stent extrusion member comprises a lumen opened at a distal end thereof, the sensor device accommodates a device tube accommodated in the lumen and capable of protruding from the opening of the stent extrusion member, and an operation wire interlocked with at least the pressing portion and a rear end portion of the pressing portion in the device tube, the pressing portion is exposed from the device tube by movement of the device tube toward the rear end side or pushing of the operation wire, and the sensor unit is brought into contact with the inner surface of the stent indwelling into the living body by the pressing portion.

(9) The stent delivery system according to any one of (1) to (8), in which a sensor unit comprises a wired sensor.

(10) The stent delivery system according to any one of (1) to (9), in which the sensor unit comprises a wireless sensor.

(11) The stent delivery system according to any one of (1) to (10), in which the sensor unit is a sensor unit configured to detect a blood velocity which is the biological-related information.

(12) The stent delivery system according to any one of (1) to (11), in which the sensor unit is a pressure sensor.

(13) The stent delivery system according to any one of (1) to (12), in which the sensor device comprises a wire-shaped strain sensor possessing the sensor unit and the wire-shaped pressing portion, and the strain sensor and the pressing portion are integrated in parallel.

(14) The stent delivery system according to any one of (1) to (13) further comprises an arithmetic processing device configured to calculate the biological-related information or the stent state information in the stent using the signal from the sensor unit.

(15) The stent delivery system according to any one of (1) to (14), in which the sensor unit is a pressure sensor, and the arithmetic processing device outputs information on blood velocity using a difference in pressure-related signal values that are detected by the same sensor unit at different positions in a radial direction of a blood vessel.

(16) The stent delivery system according to any one of (1) to (14), in which the sensor unit is a pressure sensor, and the arithmetic processing device outputs information on blood velocity using a difference in pressure-related signal values that are detected by the same sensor unit at different positions in a radial direction of a blood vessel.

(17) The stent delivery system according to any one of (1) to (15), in which the pressing portion includes a shape memory alloy.

(18) Another aspect of the disclosure here involves a combination of a stent sensor device and a stent. The stent is substantially cylindrically shaped and has a plurality of side wall openings that pass through a wall of the stent from an outer surface of the stent to an inner surface of the stent. The stent is compressed radially inwardly in a central axis direction at a time of insertion of the stent sensor device and the stent into a living body, and is expandable radially outwardly at a time of indwelling the stent sensor device and the stent into the living body. The stent sensor device is positioned inside the stent and comprises: at least one sensor unit positioned inside the stent, with the at least one type of sensor being a wire-shaped pressure sensor, a contact force sensor, or a strain sensor; and a pressing portion connected to the sensor unit to press the sensor unit toward the inner surface of the stent. The sensor device is removable from a rear end side of the stent after the stent indwells into the living body.

A stent delivery system disclosed here may include: a stent that has a plurality of side wall openings, that is formed in a substantially cylindrical shape, is compressed in a central axis direction at a time of insertion into a living body, and that is expandable outward at a time of indwelling into the living body; a sheath in which the stent is accommodated in a distal portion; and a stent extrusion member whose distal side portion is positioned in a distal side portion of the sheath. The stent delivery system is capable of releasing the stent by moving the sheath toward a rear end side with respect to the stent extrusion member. The stent delivery system further includes a sensor device. The sensor device includes: a sensor unit; and a pressing portion capable of bringing the sensor unit into contact with an inner surface of the stent indwelling into the living body. The sensor device is capable of moving the sensor unit in a rear end direction of the stent by movement of the stent extrusion member toward the rear end side or withdrawal of the sensor device. The stent delivery system is capable of obtaining biological-related information or stent state information in the stent using a signal from the sensor unit. The sensor device is capable of being taken out from the living body by removing the stent delivery system from the living body or removing the sensor device from the stent delivery system.

Therefore, in the stent delivery system, after a stent indwells in a living body, biological-related information or stent state information in the stent can be obtained, a sensor that collects the biological-related information or the stent state information can be taken out by removing the stent delivery system from the living body and/or removing the sensor device from the stent delivery system, and a problem caused by a residual sensor does not occur.

Another aspect of the disclosure here involves a method comprising: inserting a stent into a living body, the stent being substantially cylindrically shaped and having a plurality of side wall openings that pass through a wall of the stent from an outer surface of the stent to an inner surface of the stent, with the stent being compressed radially inwardly during the inserting of the stent into the living body. The inserting of the stent into the living body occurs while a sensor is positioned inside the stent. The method additionally involves radially outwardly expanding the stent after the stent is positioned in the living body; moving the sensors that are positioned inside the stent toward the inner surface of the stent, with the moving of the sensors toward the inner surface of the stent occurring after starting the radially outwardly expanding of the stent; using a signal produced by the sensor to determine information about blood flowing in the living body or information about a state of the stent; and removing the sensor from the living body while maintaining the stent in the living body.

### BRIEF DESCRIPTION OF THE DRAWINGS

FIG. 1 is a partially omitted front view of a stent delivery system according to an embodiment of the present invention.

FIG. 2 is a longitudinal cross-sectional view of the stent delivery system illustrated in FIG. 1.

FIG. 3 is an enlarged cross-sectional view of a distal portion of the stent delivery system illustrated in FIG. 1.

FIG. 4 is an enlarged cross-sectional view taken along a section line IV-IV in FIG. 3.

FIG. 5 is an enlarged cross-sectional view taken along a section line V-V in FIG. 4.

FIG. 6 is a partially omitted enlarged cross-sectional view of a rear end portion of the stent delivery system illustrated in FIG. 1.

FIG. 7 is an enlarged cross-sectional view of a distal portion of a stent delivery system according to another embodiment of the present invention.

FIG. 8 is an enlarged cross-sectional view of an example of a pressing member wire integrated member used in the stent delivery system according to the present invention.

FIG. 9 is an enlarged cross-sectional view of another example of the pressing member wire integrated member used in the stent delivery system according to the present invention.

FIG. 10 is an enlarged cross-sectional view of another example of the pressing member wire integrated member used in the stent delivery system according to the present invention.

FIG. 11 is an enlarged lateral cross-sectional view of a distal portion of a stent delivery system according to another embodiment of the present invention.

FIG. 12 is an enlarged lateral cross-sectional view of a distal portion of a stent delivery system according to another embodiment of the present invention.

FIG. 13 is a front view of an example of an in-vivo indwelling stent used in the stent delivery system according to the present invention.

FIG. 14 is a view illustrating the in-vivo indwelling stent illustrated in FIG. 13.

FIG. 15 is a view illustrating the in-vivo indwelling stent illustrated in FIG. 13.

FIG. 16 is a view illustrating an effect of the stent delivery system illustrated in FIG. 1.

FIG. 17 is a view illustrating an effect of the stent delivery system illustrated in FIG. 1.

FIG. 18 is a view illustrating an effect of the stent delivery system illustrated in FIG. 1.

FIG. 19 is a view illustrating an effect of the stent delivery system illustrated in FIG. 1.

FIG. 20 is a view illustrating an effect of the stent delivery system illustrated in FIG. 1.

FIG. 21 is an enlarged cross-sectional view of a distal portion of a stent delivery system according to another embodiment of the present invention.

FIG. 22 is an enlarged cross-sectional view of a distal portion of a stent delivery system according to another embodiment of the present invention.

FIG. 23 is an enlarged cross-sectional view of a distal portion of a stent delivery system according to another embodiment of the present invention.

FIG. 24 is an enlarged cross-sectional view of a distal portion of a stent delivery system according to another embodiment of the present invention.

FIG. 25 is an enlarged cross-sectional view of a distal portion of a stent delivery system according to another embodiment of the present invention.

FIG. 26 is an enlarged cross-sectional view taken along a section line XXVI-XXVI in FIG. 25.

FIG. 27 is a longitudinal cross-sectional view of a stent delivery system according to another embodiment of the present invention.

FIG. 28 is a longitudinal cross-sectional view of a stent delivery system according to another embodiment of the present invention.

FIG. 29 is an enlarged cross-sectional view of a distal portion of the stent delivery system illustrated in FIG. 28.

FIG. 30 is an enlarged cross-sectional view of a distal portion of a stent delivery system according to another embodiment of the present invention.

FIG. 31 is a longitudinal cross-sectional view of a stent delivery system according to another embodiment of the present invention.

FIG. 32 is an enlarged cross-sectional view of a distal portion of the stent delivery system illustrated in FIG. 31.

FIG. 33 is a view illustrating an effect of the stent delivery system illustrated in FIG. 31.

FIG. 34 is a view illustrating an effect of the stent delivery system illustrated in FIG. 31.

FIG. 35 is a view illustrating an effect of the stent delivery system illustrated in FIG. 31.

FIG. 36 is a view illustrating an effect of the stent delivery system illustrated in FIG. 31.

FIG. 37 is a view illustrating an effect of the stent delivery system illustrated in FIG. 31.

FIG. 38 is a view illustrating an effect of a stent delivery system according to another embodiment of the present invention.

FIG. 39 is a partially omitted longitudinal cross-sectional view of a stent delivery system according to another embodiment of the present invention.

FIG. 40 is a view illustrating another effect of the stent delivery system illustrated in FIG. 39.

FIG. 41 is a view illustrating another effect of the stent delivery system illustrated in FIG. 31.

FIG. 42 is a view illustrating another effect of the stent delivery system illustrated in FIG. 31.

FIG. 43 is a view illustrating another effect of the stent delivery system illustrated in FIG. 31.

FIG. 44 is a view illustrating another effect of the stent delivery system illustrated in FIG. 31.

FIG. 45 is a view illustrating a stent delivery device using the stent delivery system according to the present invention.

FIG. 46 is a view illustrating an effect of the stent delivery device illustrated in FIG. 45.

FIG. 47 is a view illustrating an effect of the stent delivery device illustrated in FIG. 45.

### DETAILED DESCRIPTION

Set forth below with reference to the accompanying drawings is a detailed description of embodiments of a stent delivery system and a stent sensor device representing examples of the new stent delivery system and stent sensor device disclosed here.

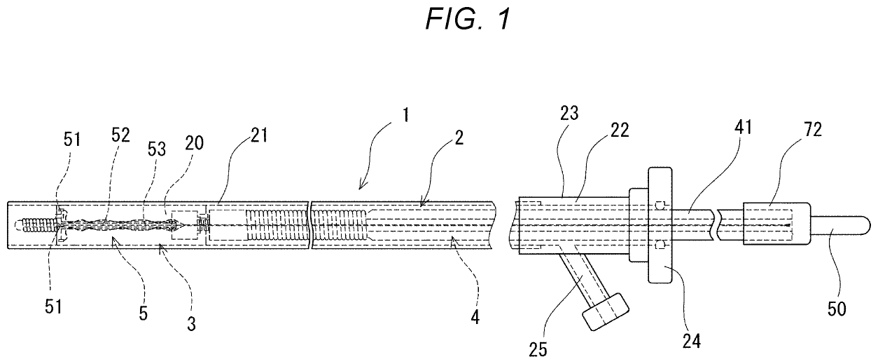

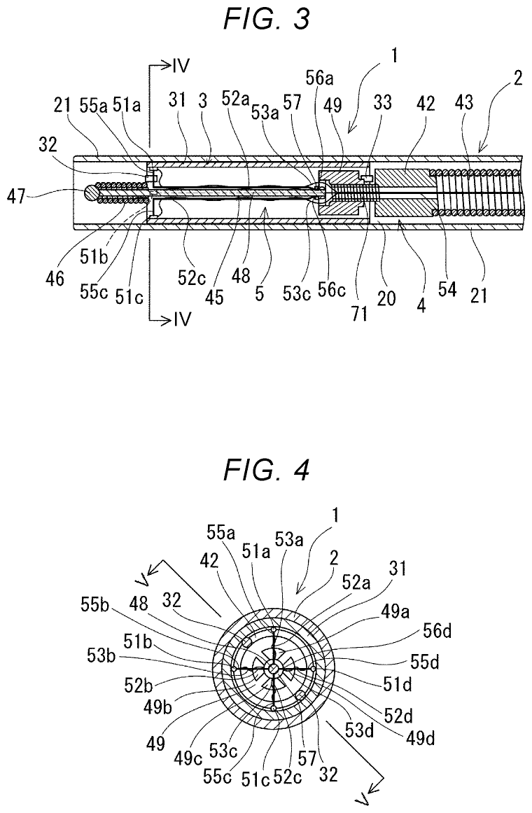

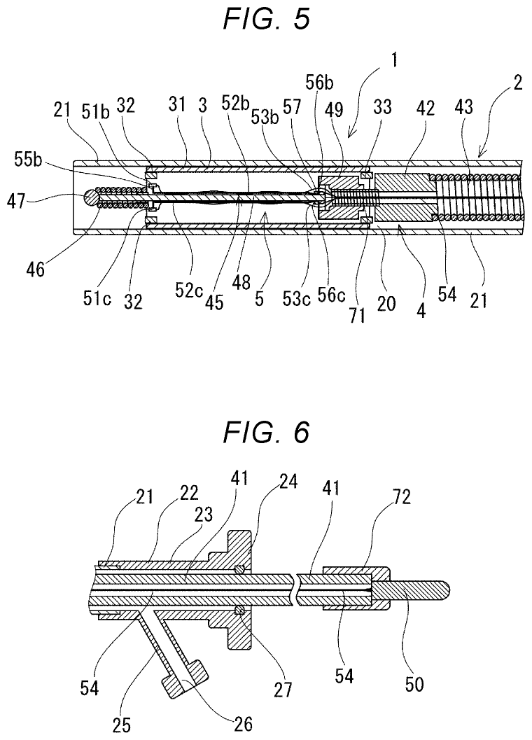

A stent delivery system 1 according to one embodiment includes: a stent 3 that has a plurality of side wall openings, that is formed in a substantially cylindrical shape and compressed in a central axis direction at the time of insertion into a living body, and that is expandable outward at the time of indwelling into the living body; a sheath (stent accommodation tube) 21 in which the stent 3 is accommodated in a distal portion of the sheath; and a stent extrusion member (stent extrusion member 4 in an embodiment illustrated in FIGS. 1 to 25) whose distal side portion is located in a distal side portion of the sheath 21. The stent delivery system 1 is capable of releasing the stent 3 by moving the sheath 21 toward a rear end side with respect to the stent extrusion member.

The stent delivery system 1 includes a sensor device 5 (sensor). The sensor device 5 includes: a sensor unit; and a pressing portion that brings the sensor unit into contact with an inner surface of the stent 3 indwelling into the living body. Further, the sensor device 5 is capable of moving the sensor unit in a rear end direction of the stent 3 by movement of the stent extrusion member toward a rear end side or retraction (withdrawal or pulling) of the sensor device 5. The stent delivery system 1 can obtain biological-related information or stent state information in the stent 3 using a signal from the sensor unit. The biological-related information is information related to a living body, for example, information related to a liquid pressure, a blood velocity, and the like of blood in a blood vessel. The stent state information is information related to a placement state of the stent, and is, for example, information related to a shape of the stent indwelling in the blood vessel and stent malapposition (the stent indwells in a blood vessel in an abnormal state such as a state where the stent or part of the stent is floated or spaced from a vascular wall). The sensor device 5 can be taken out from the living body by removing the stent delivery system 1 from the living body or removing the sensor device 5 from the stent delivery system 1.

The stent delivery system 1 according to an embodiment illustrated in FIGS. 1 to 6 will be described.

As illustrated in FIGS. 1 to 6, the stent delivery system 1 according to this embodiment includes a tube assembly 2 including the stent accommodation tube 21, a self-expandable stent 3 accommodated in the stent accommodation tube 21, the stent extrusion member 4 slidably accommodated in the tube 21, and the sensor device 5 disposed in the stent 3.

The stent accommodation tube 21 is a tube-shaped body (tubular body), and a distal end and a rear end of the tube 21 are open. A distal end opening functions as a discharge port of the stent 3 when the stent 3 indwells in a stenosed site in a body-cavity. The stent accommodation tube 21 is slid toward the rear end side, so that the stent 3 is released from the distal end opening, a stress load is released, and the stent 3 is expanded and returns to a shape before compression. The distal portion of the stent accommodation tube 21 is a stent accommodation portion that accommodates the stent 3 therein.

An outer diameter of the stent accommodation tube 21 is preferably about 0.4 mm to 4.0 mm, and particularly preferably 0.5 mm to 3.0 mm. An inner diameter of the stent accommodation tube 21 is preferably about 0.3 mm to 2.0 mm. The stent accommodation tube 21 is preferably a flexible tube. For the stent accommodation tube 21, a material having a certain degree of flexibility, for example, a thermoplastic resin such as polyolefin (for example, polyethylene, polypropylene, ethylene-propylene copolymer, and ethylene-vinyl acetate copolymer, and further including a crosslinked or partially crosslinked product), polyvinyl chloride, polyamide elastomer, or polyurethane, silicone rubber, or latex rubber can be used, and the thermoplastic resin described above is preferable.

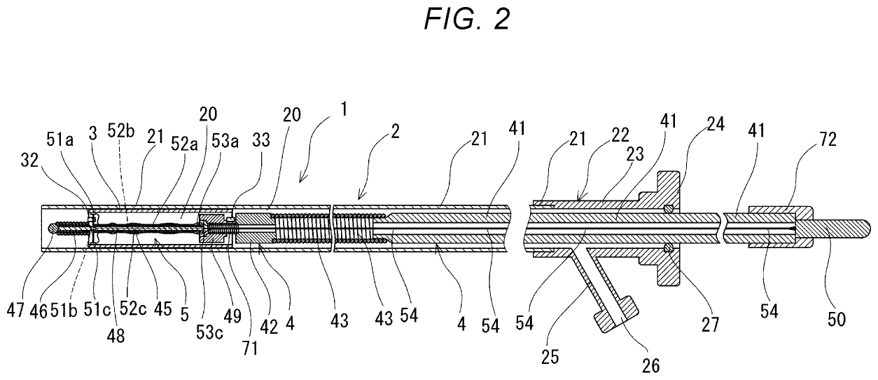

As illustrated in FIGS. 1 and 2, the tube assembly 2 includes a lumen 20 therein and a tube hub 22 fixed to a rear end portion of the stent accommodation tube 21. The tube hub 22 includes a tube hub main body 23 and a seal member 27 accommodated in a rear end flange portion 24 of the tube hub main body 23 and holding the stent extrusion member 4 in a slidable and liquid-tight manner. The tube hub 22 includes a branch port portion 25 branched obliquely rearward from a vicinity of a center of the tube hub main body 23. An opening portion 26 of the branch port portion 25 is in communication with the inside of the stent accommodation tube 21.

As illustrated in FIGS. 1 to 6, the stent extrusion member 4 includes a hollow shaft portion 41, a distal end guiding portion 45, an elastic tubular portion 43 provided at a distal end of the hollow shaft portion 41, a stent pressing portion 42 provided at a distal end of the elastic tubular portion 43, a guide portion fixing portion 49 provided at a rear end of the distal end guiding portion 45, and an elastic tubular interlock portion 71 that interlocks the stent pressing portion 42 and the guide portion fixing portion 49.

The distal end guiding portion 45 includes a guide portion shaft 48, a spherical distal portion 47 fixed to a distal end of the guide portion shaft 48, and a distal coil member 46 extending from a rear portion of the spherical distal portion 47 in a rear end direction of the guide portion shaft 48. The distal coil member 46 has a distal end fixed to the rear portion of the spherical distal portion 47 and a rear end fixed to a distal portion side portion of the guide portion shaft 48. In this embodiment, a distal side of the distal coil member 46 protrudes from a distal end of the stent 3 with respect to a rear end portion of the distal coil member 46. The guide portion shaft 48 is a solid shaft portion. The distal coil member 46 is preferably a coil spring.

Since the guide portion shaft 48 and the spherical distal portion 47 are also a distal portion of the stent delivery system 1, it is preferable that positions of the guide portion shaft 48 and the spherical distal portion 47 can be easily checked under X-ray fluoroscopy. It is preferable to use Pt, a Pt alloy (for example, Pt—Ir alloy), W, a W alloy, Ag, an Ag alloy, or the like as the material from which the distal end guiding portion 45 is fabricated.

A rear end portion of the guide portion shaft 48 is inserted into and fixed to a central opening portion of the guide portion fixing portion 49. A coil spring extending for a predetermined length is used as the elastic tubular portion 43 provided at the distal end of the hollow shaft portion 41. A coil spring is used as the elastic tubular interlock portion 71 that interlocks the stent pressing portion 42 and the guide portion fixing portion 49. The coil spring used as the elastic tubular interlock portion 71 has a smaller diameter and is shorter than the coil spring used as the elastic tubular portion 43.

The stent pressing portion 42 and the guide portion fixing portion 49 preferably have X-ray contrast properties, and the entire body or surfaces of the stent pressing portion 42 and the guide portion fixing portion 49 are preferably formed of an X-ray contrast material. The stent 3 to be described later includes a stopper 33 provided at a rear end portion, and the stopper 33 is located between the stent pressing portion 42 and the guide portion fixing portion 49. Further, a distal end of the stent pressing portion 42 is attachable to a rear end of the stopper 33. A rear end of the guide portion fixing portion 49 is attachable to a distal end of the stopper 33. A shaft hub 72 is fixed to a rear end of the hollow shaft portion 41.

As illustrated in FIGS. 1 to 5, the stent delivery system 1 according to this embodiment includes the sensor device 5. The sensor device 5 includes sensor units or sensors 51 (51a, 51b, 51c, and 51d), pressing portions 52 (52a, 52b, 52c, and 52d) that bring the sensor units 51 (51a, 51b, 51c, and 51d) into contact with an inner surface of the stent 3 indwelling into the living body, and sensor cables 53 (53a, 53b, 53c, and 53d) whose distal ends are electrically connected to the sensor units 51 (51a, 51b, 51c, and 51d). The sensor unit or sensors 51 (51a, 51b, 51c, and 51d) are wired sensors.

The sensor device 5 according to this embodiment and a sensor device to be described later are also stent sensor devices according to the present disclosure.

The stent sensor device 5 h is disposed inside the stent 3, which has a plurality of side wall openings, is formed in a substantially cylindrical shape, is compressed in a central axis direction at the time of insertion into the living body, and which can expand outward and return to a shape before compression at the time of indwelling into the living body. The sensor device includes at least one type of sensor unit 51 selected from a wire-shaped pressure sensor, a contact force sensor, or a strain sensor, and the pressing portions 52 that press the sensor units 51 to the inside of the stent. After the stent indwells into the living body, the sensor device can be removed from a rear end side of the stent 3.

The sensor device 5 can move the sensor units in a rear end direction of the stent 3 by the movement of the stent extrusion member (stent extrusion member 4) toward a rear end side or the retraction (withdrawal or pulling) of the sensor device 5.

The stent delivery system 1 according to this embodiment can obtain biological-related information or stent state information using signals from the sensor units.

As the sensor units 51 (51a, 51b, 51c, and 51d), a pressure sensor, a contact force sensor, a strain sensor, or the like is used. As the pressure sensor, for example, a thin-film type pressure sensor or an extra-fine diameter optical fiber pressure sensor can be used.

In the stent delivery system 1 according to this embodiment, as illustrated in FIGS. 1 to 5, the sensor units 51 (51a, 51b, 51c, and 51d) are pressed into contact with the inner surface of the stent 3 by the pressing portions 52 (52a, 52b, 52c, and 52d) whose distal ends are fixed to the sensor units 51 (51a, 51b, 51c, and 51d). Further, as illustrated in FIGS. 16 to 20, the sensor units 51 (51a, 51b, 51c, and 51d) can be pressed (attached), by the pressing portions 52 (52a, 52b, 52c, and 52d), against the inner surface of the stent 3 indwelling into the living body. The pressing portions 52 (52a, 52b, 52c, and 52d) can press (attach), with substantially equal pressures, the attached sensor units 51 (51a, 51b, 51c, and 51d) against the inner surface of the stent 3 indwelling into the living body.

In the sensor device 5 according to this embodiment, each pressing portion is a wire-shaped (elongated) pressing portion. Specifically, a linear main body portion and bent portions 55a, 55b, 55c, and 55d bent from the main body portion are provided. The sensor units are fixed to distal ends of the bent portions 55a, 55b, and 55d of the pressing portions.

As the pressing portions 52 (52a, 52b, 52c, and 52d), an elastic linear body (elastic wire) having a spring property capable of pressing the sensor units 51 (51a, 51b, 51c, and 51d) is used. The pressing portions 52 (52a, 52b, 52c, and 52d) may not have a strong spring property as long as the sensor units 51 (51a, 51b, 51c, and 51d) can be pressed into contact with the inner surface of the stent. The pressing portions may include a shape memory alloy in order to have a spring property.

In this embodiment, in a state illustrated in FIGS. 1 to 5, the pressing portions 52 (52a, 52b, 52c, and 52d) are pressed by the sensor units, which are pressed by the stent 3, and are elastically deformed inward. In this embodiment, as illustrated in FIGS. 1 to 5, rear end portions of the pressing portions 52 (52a, 52b, 52c, and 52d) are fixed to the rear end portion of the guide portion shaft 48 or a distal portion of the guide portion fixing portion 49. In this embodiment, rear end portions 56a, 56b, 56c, and 56d of the pressing portions 52 (52a, 52b, 52c, and 52d) are located and fixed between the rear end portion of the guide portion shaft 48 and a ring-shaped fixing portion 57.

The distal ends of the sensor cables 53 (53a, 53b, 53c, and 53d) are electrically connected to the sensor units 51 (51a, 51b, 51c, and 51d). The sensor cables 53 (53a, 53b, 53c, and 53d) pass through the stent accommodation tube 21, enter the stent extrusion member 4 from four opening portions 49a, 49b, 49c, and 49d formed in the guide portion fixing portion 49 through the distal portion of the guide portion fixing portion 49, pass through the guide portion fixing portion 49, the elastic tubular interlock portion 71, the stent pressing portion 42, the elastic tubular portion 43, and the hollow shaft portion 41, and are electrically connected to a connector 50 located at the rear end of the hollow shaft portion 41. In this embodiment, the sensor cables 53 (53a, 53b, 53c, and 53d) are bundled or twisted to form a single wire body 54 in the stent extrusion member 4, specifically, in the guide portion fixing portion 49, and the single wire body 54 extends in the stent extrusion member 4 and is electrically connected to the connector 50.

In this embodiment, the connector 50 is fixed to the shaft hub 72, and when the sensor device is pulled, the user also pulls the stent extrusion member 4 together because the sensor device is fixed to the shaft hub 72 which is fixed to the stent extrusion member 4. In this embodiment, the sensor cables 53 (53a, 53b, 53c, and 53d) are wound around the pressing portions 52 (52a, 52b, 52c, and 52d) at a distal side portion.

In all the embodiments to be described later, the sensor device includes both a sensor cable and a pressing portion, and the sensor cable is not limited to a type in which the sensor cable is wound around the pressing portion as described above.

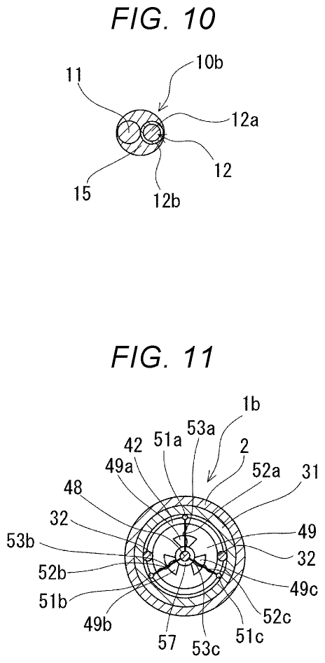

For example, as illustrated in FIG. 8, a sensor cable 12 including a conductive wire 12a and a coating film 12b may be disposed on a side surface of a pressing portion 11, and the sensor cable 12 and the pressing portion 11 may be fixed to each other by an adhesive portion 13 to form an integrated member 10. For example, as illustrated in FIG. 9, the sensor cable 12 including the conductive wire 12a and the coating film 12b may be disposed on the side surface of the pressing portion 11, and the integrated member 10a may be formed by a covering tube 14 that covers and fixes both the sensor cable 12 and the pressing portion 11. Further, as illustrated in FIG. 10, the sensor cable 12 including the conductive wire 12a and the coating film 12b may be disposed on the side surface of the pressing portion 11, and the integrated member 10b may be formed by a resin layer 15 that covers and fixes both the sensor cable 12 and the pressing portion 11.

In this embodiment, as illustrated in FIGS. 1 to 6, particularly in FIG. 4, the sensor device 5 includes two or more sensor units (sensors) spaced apart by a predetermined distance. Specifically, it is preferable to include three or more sensor units. When three or more sensor units are provided, it is preferable that the sensor units are attached, in a manner of being annular and spaced apart by a predetermined distance, to the inner surface of the stent 3 indwelling into the living body. In the embodiment illustrated in FIGS. 1 to 6, particularly in FIG. 4, four sensor units are provided, and as illustrated in FIG. 16, the sensor units are attached, in a manner of being annular and spaced apart by a predetermined distance, to the inner surface of the stent 3 indwelling into the living body.

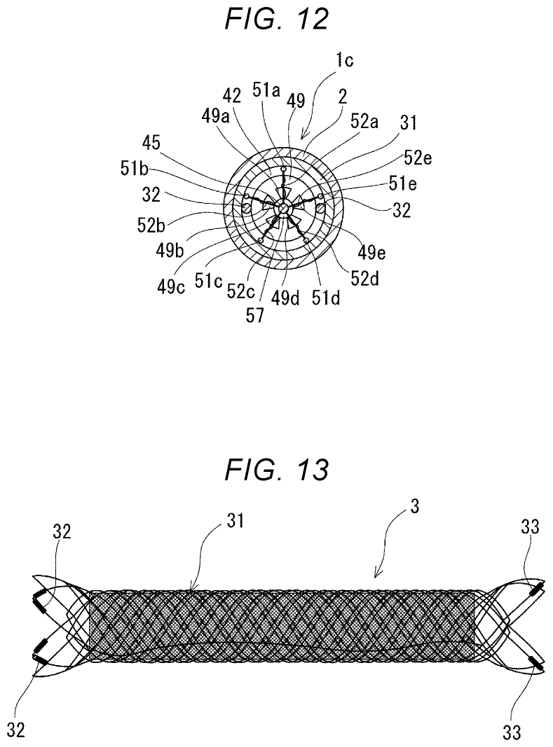

The number of sensor units in the sensor device is not limited to four as described above, and may be three as in a stent delivery system 1b according to an embodiment illustrated in FIG. 11. In this case, it is preferable that the sensor units are also attached, in a manner of being annular and spaced apart by a predetermined distance, to the inner surface of the stent 3 indwelling into the living body. The number of sensor units may be five as in a stent delivery system 1c according to an embodiment illustrated in FIG. 12. In this case, it is preferable that the sensor units are also attached, in a manner of being annular and spaced apart by a predetermined distance, to the inner surface of the stent 3 indwelling into the living body. In the stent delivery system 1c according to this embodiment, the sensor device includes the sensor units 51 (51a, 51b, 51c, 51d, and 51e), the pressing portions 52 (52a, 52b, 52c, 52d, and 52e) that press the sensor units against the inner surface of the stent 3 indwelling into the living body, and the sensor cables 53 (53a, 53b, 53c, 53d, and 53e) electrically connected to the sensor units. Further, the guide portion fixing portion 49 of the stent extrusion member 4 has five opening portions 49a, 49b, 49c, 49d, and 49e through which the sensor cables 53 (53a, 53b, 53c, 53d, and 53e) pass.

In the embodiment illustrated in FIGS. 1 to 6, as illustrated in FIGS. 16 to the sensor units 51 (51a, 51b, 51c, and 51d) of the sensor device 5 are movable in the rear end direction of the stent 3 in a state where the sensor units are attached, in a manner of being annular and spaced apart by a predetermined distance, to the inner surface of the stent 3 indwelling into the living body by the movement of the stent extrusion member (stent extrusion member 4) toward the rear end side. Therefore, it is possible to obtain biological-related information or stent state information in the stent 3 at a plurality of positions in the stent 3 using signals from the sensor units.

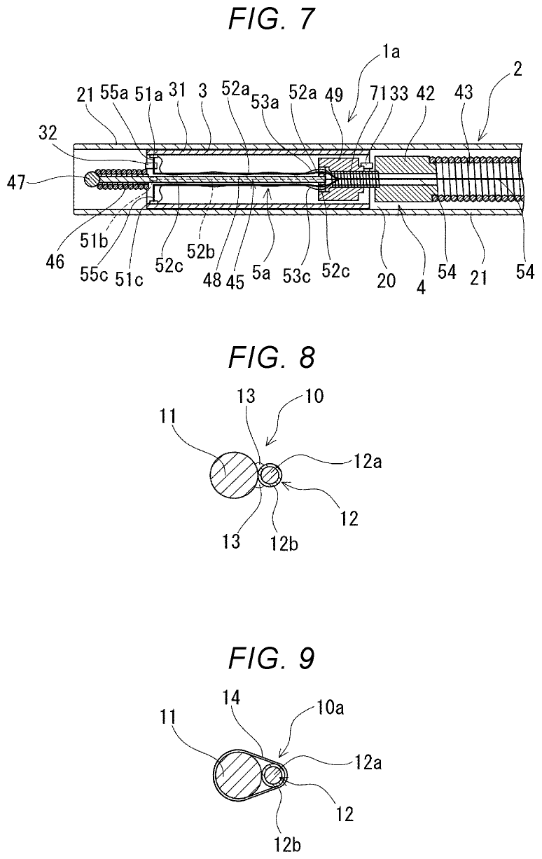

In the stent delivery system 1 according to the embodiment illustrated in FIGS. 1 to 6, the pressing portions 52 (52a, 52b, 52c, and 52d) of the sensor device are fixed to the guide portion fixing portion 49 of the stent extrusion member 4, but are not limited to this type. For example, as in a sensor device 5a of a stent delivery system 1a illustrated in FIG. 7, the pressing portions 52 (52a, 52b, 52c, and 52d) may not be fixed to the guide portion fixing portion 49 of the stent extrusion member 4. In particular, in the stent delivery system 1a according to this embodiment, rear ends of the pressing portions 52 (52a, 52b, 52c, and 52d) enter the guide portion fixing portion 49 from an opening portion of the guide portion fixing portion 49 and are bundled. Furthermore, in the stent delivery system 1a according to this embodiment, the connector 50 electrically connected to the sensor cables is fixed to the shaft hub 72, and the shaft hub 72 is not fixed to the rear end of the hollow shaft portion 41. Therefore, when the connector 50 is pulled rearward together with the shaft hub 72, a distal portion of the sensor device moves rearward, and the pressing portions 52 (52a, 52b, 52c, and 52d) can be accommodated in the guide portion fixing portion 49 of the stent extrusion member 4. In this embodiment, the sensor cables 53 (53a, 53b, 53c, and 53d) are bundled to form a traction wire, meaning the user can move the sensor device by withdrawing the sensor cables 53. Thus, the bundle of the sensor cables serve as a traction wire allowing retraction or withdrawal of the sensor device. A traction wire may be separately provided, and the sensor cables 53 (53a, 53b, 53c, and 53d) may be wound around the traction wire. When the traction wire is separately provided, the rear ends of the pressing portions 52 (52a, 52b, 52c, and 52d) are preferably fixed to a distal end of the traction wire. The sensor units 51 (51a, 51b, 51c, and 51d) may also be accommodated in the guide portion fixing portion 49.

As the stent 3, a so-called self-expandable stent that has a plurality of side wall openings, that is formed in a substantially cylindrical shape, that is compressed in the central axis direction (radially inwardly) at the time of insertion into the living body, and that can expand outward and return to a shape before compression at the time of indwelling into the living body is used.

Any stent may be used as the self-expandable stent, and for example, the stent 3 as illustrated in FIGS. 13 to 15 can be suitably used. The stent 3 according to this embodiment is effective for ameliorating an aneurysm forming portion of a cerebral blood vessel.

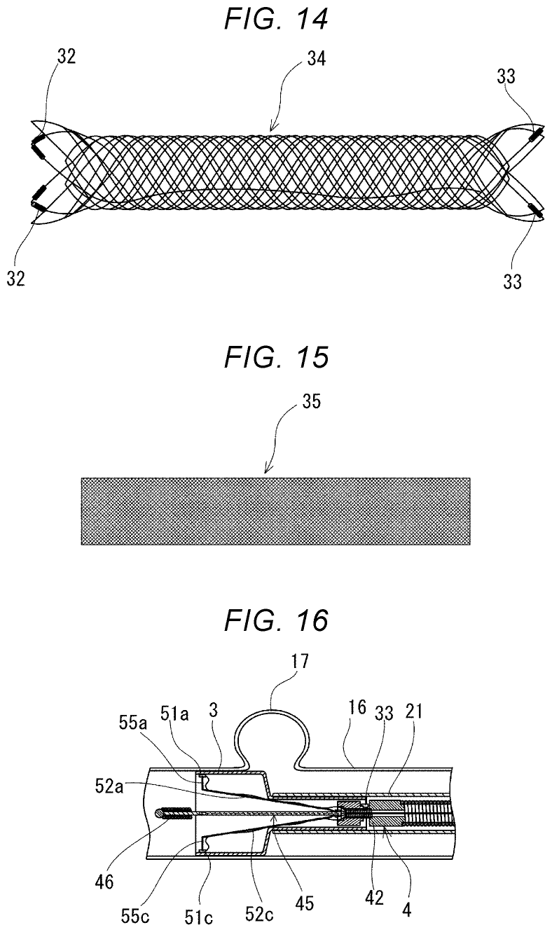

For example, when the stent 3 indwells in the cerebral blood vessel, a diameter of the stent 3 at the time of expansion (non-compression) is preferably about 0.5 mm to 6.0 mm, and particularly more preferably 0.9 mm to 5.0 mm. A length of the stent at the time of expansion (non-compression) is preferably about 5 mm to 50 mm. A thickness of the stent is preferably about 0.05 mm to 0.15 mm, and particularly more preferably 0.06 mm to 0.13 mm. FIGS. 13 and 14 also show an example of side wall openings passing through the wall of the stent.

As a constituent material from which the stent may be fabricated, a superelastic metal is preferred. As the superelastic metal, a superelastic alloy is preferably used. The superelastic alloy referred to herein is generally called a shape memory alloy, and exhibits superelasticity at least at a biological temperature (around 37° C.). Particularly preferably, a superelastic alloy such as a Ti—Ni alloy of 49 atom % to 53 atom % of Ni, a Cu—Zn alloy of 38.5 weight % to 41.5 weight % of Zn, a Cu-Zn—X alloy of 1 weight % to 10 weight % of X (X=Be, Si, Sn, Al, Ga), an Ni—Al alloy of 36 atom % to 38 atom % of Al, and an Mg—Sc alloy of 15 atom % to 25 atom % of Sc is preferably used. Particularly preferably, the above Ti—Ni alloy is used. Mechanical properties can be appropriately changed by preparing a Ti—Ni—X alloy (X=Co, Fe, Mn, Cr, V, Al, Nb, W, B, etc.) in which a part of the Ti—Ni alloy is substituted with 0.01% to 10.0% of X, preparing a Ti—Ni—X alloy (X=Cu, Pb, Zr) in which a part of the Ti—Ni alloy is substituted with 0.01% to 30.0% of X, and selecting a cold working ratio and/or conditions of final heat treatment. The mechanical properties can be appropriately changed by selecting the cold working ratio and/or the conditions of the final heat treatment using the Ti—Ni—X alloy described above. A buckling strength (yield stress when loaded) of the superelastic alloy to be used is 5 kg/mm² to 200 kg/mm² (at 22° C.), preferably 8 kg/mm² to 150 kg/mm², and restoring stress (yield stress when unloaded) of the superelastic alloy is 3 kg/mm² to 180 kg/mm² (at 22° C.), more preferably 5 kg/mm² to 130 kg/mm². The term “superelasticity” here means that, even when a normal metal is deformed (bent, stretched, or compressed) to a region where the normal metal is plastically deformed at an operating temperature, the metal substantially returns to a shape before compression without requiring heat after the deformation is released.

The stent 3 according to this embodiment includes a stent main body 31, a distal side marker 32, and a rear end side stopper 33. In the stent according to this embodiment, both the distal side marker 32 and the rear end side stopper 33 are formed of coil springs. It is preferable that positions of the distal side marker 32 and the rear end side stopper 33 can be easily checked under X-ray fluoroscopy, and Pt, a Pt alloy (for example, a Pt—Ir alloy), W, a W alloy, Ag, an Ag alloy, or the like is preferably used as the material of the distal side marker 32 and the rear end side stopper 33.

The stent 3 according to this embodiment is a double-layered stent, and the stent main body 31 is double-layered. The stent main body 31 includes a tubular stent outer layer 34 as illustrated in FIGS. 13 and 14, and a tubular stent inner layer 35 disposed inside the stent outer layer 34 as illustrated in FIGS. 13 and 15.

The stent outer layer 34 is formed in a tubular shape by a wire braid, and includes the distal side marker 32 and the rear end side stopper 33 described above at both end portions. The stent inner layer 35 is formed in a tubular shape by a wire braid that is thinner than the stent outer layer 34, and is a tight tubular body. Therefore, a side wall opening formed in a tubular side surface of the stent inner layer 35 is sufficiently smaller than a side wall opening formed in a tubular side surface of the stent outer layer 34.

The stent inner layer 35 is shorter than the stent outer layer 34. That is, both end portions of the stent main body 31 are formed only by the stent outer layer 34, and the stent inner layer 35 is not present. The distal side marker 32 and the rear end side stopper 33 described above are provided at the end portions of the stent main body 31 formed only by the stent outer layer 34.

In the stent 3 according to this embodiment, the stent inner layer 35 functions as a flow-diverting layer for changing a direction of blood flow. The stent inner layer 35 that functions as the flow-diverting layer is preferably formed in a tubular shape by braiding a fine-diameter superelastic metal wire. As a size of the side wall opening formed in the tubular side surface of the stent inner layer 35 at the time of stent expansion, a pore diameter thereof is preferably 0.3 mm or less and an opening area thereof is preferably 0.07 mm² or less. As the size of the side wall opening of the stent inner layer 35 at the time of the stent expansion, the pore diameter thereof is preferably 0.03 mm or more and the opening area is preferably mm² or more. Both end portions of the stent inner layer 35 are preferably formed by laser cutting or etching a thin tube. A porosity of the tubular side surface of the stent inner layer 35 at the time of the stent expansion is preferably 45% to 70%.

It is preferable that the stent 3 according to this embodiment, which is a double-layered stent, has a larger radial force (defined as a radial force applied in compression in a radial direction of about 50% of the stent) than either the stent outer layer 34 or the stent inner layer 35 alone. In particular, the radial force of the stent 3 is preferably larger than a sum of the radial forces of the stent outer layer 34 and the stent inner layer 35.

A plurality of fixing portions (not illustrated) that connect the stent outer layer 34 and the stent inner layer 35 are provided at both end portions of the stent inner layer 35. The fixing portions can be formed by winding fine-diameter wires. The fixing portions can also be formed by pressing portions of the stent outer layer 34 corresponding to positions on both end portions of the stent inner layer 35. Furthermore, the fixing portions can also be formed by weaving both end portions of the stent inner layer 35 into the stent outer layer 34.

A method for using the stent delivery system 1 according to the embodiment illustrated in FIGS. 1 to 6 will be described with reference to FIGS. 16 to 20.

After the stent 3 is placed in the living body (for example, in a cerebral artery 16) in which the stent 3 is to indwell, specifically, after a distal side portion (spherical distal portion 47 and distal coil member 46) of the distal end guiding portion 45 of the stent delivery system 1 is placed in a manner of being located a predetermined distance forward (peripheral side at the predetermined distance) from a target in-vivo indwelling portion, specifically, an aneurysm 17 of the cerebral artery 16 having the aneurysm 17, by advancing the stent extrusion member 4 or retracting the stent accommodation tube 21 (tube assembly 2), the stopper 33 at the rear end portion of the stent 3 is attached to (contacted by) and pressed by a distal end surface of the stent pressing portion 42 of the stent extrusion member 4, and the stent 3 is discharged from the stent accommodation tube 21.

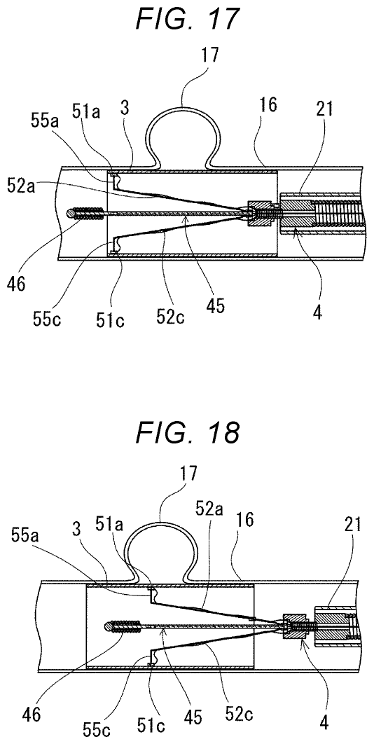

As illustrated in FIG. 16, a distal portion of the stent 3 discharged from the stent accommodation tube 21 self-expands and is pressed into contact with an inner surface of the living body, specifically, an inner surface of the cerebral artery 16. At the same time, distal side portions of the pressing portions 52 (52a, 52b, 52c, and 52d) of the sensor device 5 are also outside the stent accommodation tube 21 and spread, and the sensor units 51 (51a, 51b, 51c, and 51d) are attached to the inner surface of the expanded stent 3 (stent main body 31). By advancing the stent extrusion member 4 or retracting the stent accommodation tube 21 (tube assembly 2), as illustrated in FIG. 17, the entire stent 3 is discharged from the stent accommodation tube 21 and indwells in a manner of covering the inside of the cerebral artery 16 and an opening portion between the aneurysm 17 and the blood vessel.

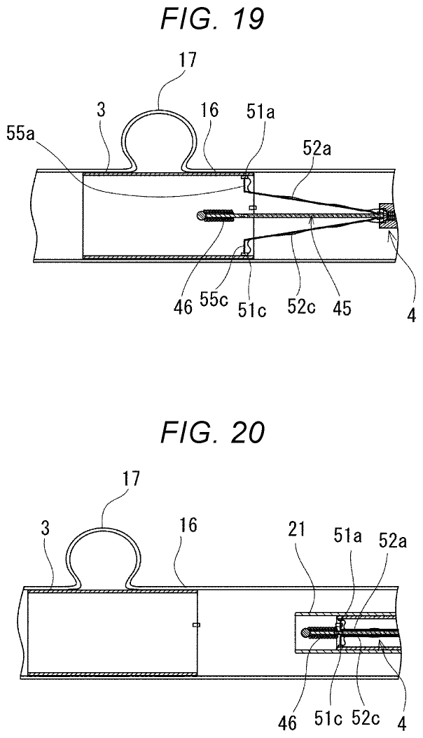

In a state illustrated in FIG. 16 and a state illustrated in FIG. 17, biological-related information or stent state information in the stent 3 (stent main body 31) can be obtained using the signals from the sensor units. For example, when a pressure sensor or a contact force sensor is used as the sensor units 51 (51a, 51b, 51c, and 51d), stent state information can be obtained (i.e., information about the state of the stent can be obtained). In the stent delivery system 1 according to this embodiment, by retracting the stent delivery system 1 from the state illustrated in FIG. 17, as illustrated in FIGS. 18 and 19, axial positions of the sensor units 51 (51a, 51b, 51c, and 51d) in the stent 3 (stent main body 31) can be changed, and stent state information can be obtained at a plurality of sites. In this embodiment, as illustrated in FIG. 20, after information collection is completed, the sensor device 5 can be accommodated in the stent accommodation tube 21 by retracting the stent extrusion member 4 with respect to the stent accommodation tube 21.

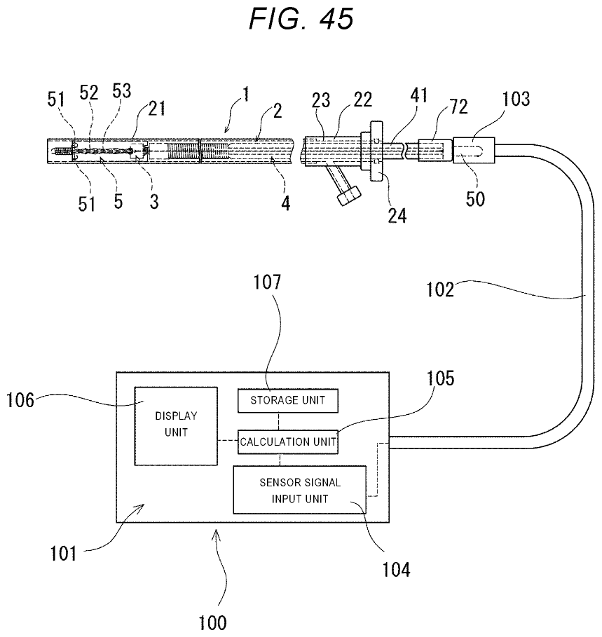

The stent delivery system 1 preferably includes an arithmetic processing device 100 (computer) as illustrated in FIG. 45. The arithmetic processing device 100 according to this embodiment includes a device main body portion 101, a cable 102 connected to the device main body portion 101, and a coupler 103 provided at a distal end of the cable 102 and connectable to the connector 50 of the stent delivery system 1. The device main body portion 101 includes a sensor signal input unit 104, a calculation unit 105 that performs calculation using a signal from the sensor signal input unit 104, a display unit 106 that performs output based on a calculation result, and a storage unit 107 that stores input data, output data, and the like.

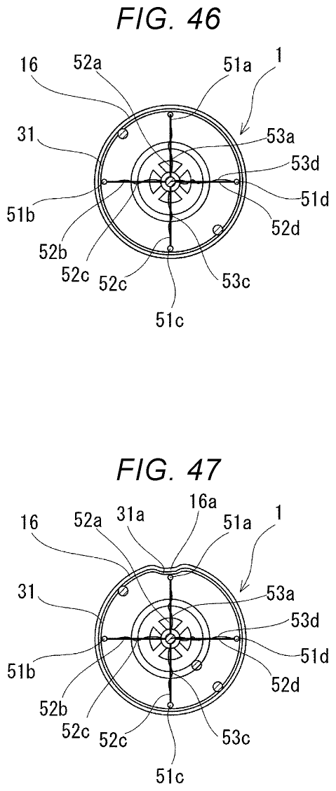

When the stent 3 is expanded in a favorable state at the in-vivo indwelling portion, a state as illustrated in FIG. 46 is obtained. Since the pressing portions 52a, 52b, 52c, and 52d press the respective sensor units 51a, 51b, 51c, and 51d against the inner surface of the stent 3 (stent main body 31) with substantially the same contact force (pressing force), signals (data) caused by the contact force to be output are also substantially the same.

The arithmetic processing device 100 according to this embodiment has a function of displaying, on the display unit 106, a value of the contact force in the sensor units 51a, 51b, 51c, and 51d calculated using the signals from the sensor units. The calculation unit 105 of the arithmetic processing device 100 outputs that a stent state is favorable when the value of the contact force in the sensor units 51a, 51b, 51c, and 51d calculated using the signals from the sensor units is within a predetermined range.

In a case where an expanded state of the stent 3 is not favorable, for example, a state as illustrated in FIG. 47 is considered. In a state illustrated in FIG. 47, the stent 3 (stent main body 31) includes a stent deformation portion 31a which is pressed by a deformation portion 16a of the cerebral artery (blood vessel) 16 and which is curved inward. The sensor unit 51a is attached to the stent deformation portion 31a. Since the sensor unit 51a is more strongly pressed inward than other sensor units 51b, 51c, and 51d, a signal (data) caused by the contact force (pressing force) to be output is different from a signal from the sensor unit 51a. A value of the contact force in the sensor units calculated using the signal from the sensor unit 51a displayed on the display unit 106 is different from (specifically, larger than the value of the contact force) the value of the contact force in the other sensor units 51b, 51c, and 51d calculated using the signals from the other sensor units. When a difference between the value of the contact force in the sensor unit 51a and the value of the contact force in the sensor units 51b, 51c, and 51d calculated using the signals from the sensor units is out of the predetermined range, the calculation unit 105 of the arithmetic processing device 100 outputs that the stent state is not favorable. When it is detected that the stent state is not favorable (in the case as that in FIG. 47), after the stent delivery system 1 is removed from the living body, a balloon catheter (not illustrated) is inserted into the stent 3, and a balloon is inflated. Accordingly, a shape of the stent 3 can be improved.

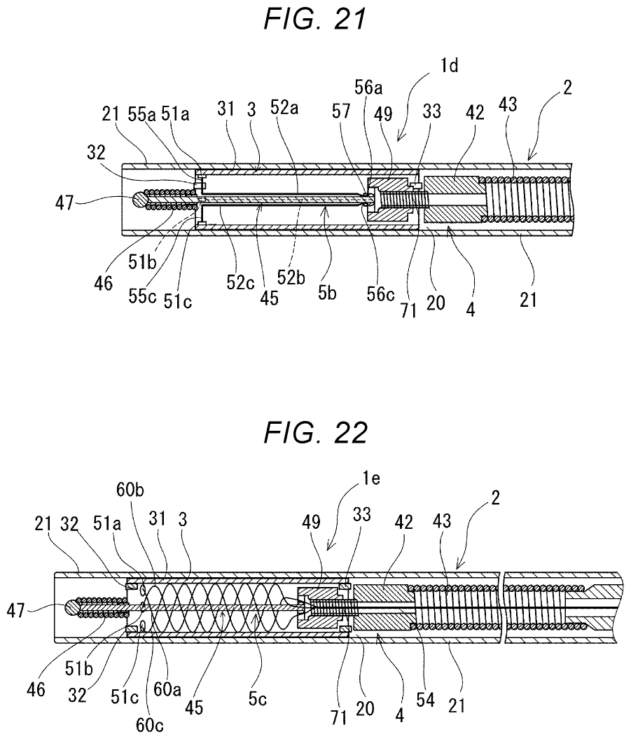

Next, a stent delivery system 1d according to an embodiment illustrated in FIG. 21 will be described. In the stent delivery system 1d according to this embodiment, a wireless sensor having a communication function as a sensor unit is used in a sensor device 5b. Therefore, although the sensor device 5b includes the sensor units 51 (51a, 51b, 51c, and 51d) and the pressing portions 52 (52a, 52b, 52c, and 52d) that press the respective sensor units on the inner surface of the stent 3 indwelling in the living body, the sensor cable is not provided. Other configurations, features and aspects of the stent delivery system 1d are the same as those of the above-described stent delivery system 1.

Next, a stent delivery system 1e according to an embodiment illustrated in FIG. 22 will be described. In the stent delivery system 1e according to this embodiment, a sensor device 5c includes wire-shaped pressing portions 60a, 60b, and 60c each having a helical shape wire, and the sensor units 51a, 51b, and 51c located at respective distal portions of the pressing portions. Specifically, in the stent delivery system 1e according to this embodiment, the sensor device 5c includes the sensor units 51 (51a, 51b, and 51c), and the pressing portions that press the sensor units are formed by spiral wires 60a, 60b, and 60c instead of linear wires as in the above-described embodiment. The spiral wires 60a, 60b, and 60c are compressibly braided (i.e., the spiral wires are braided such that the braided wires can be compressed), and when the stent 3 is expanded, the spiral wires 60a, 60b, and 60c expand and press the sensor units 51 (51a, 51b, and 51c) fixed to distal portions of the spiral wires 60a, 60b, and 60c against the inner surface of the stent 3. In the sensor device 5c according to this embodiment, the sensor cables may be wound around the spiral wires 60a, 60b, and 60c, and it is preferable to use integrated members 10a, 10b, and 10c in which the pressing portions and the sensor cables illustrated in FIGS. 8 to 10 are integrated.

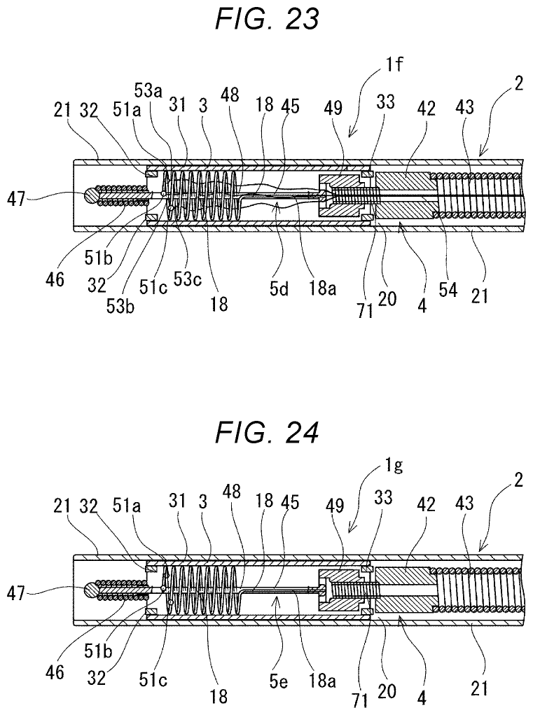

Next, a stent delivery system 1f according to an embodiment illustrated in FIG. 23 will be described. In the stent delivery system 1f according to this embodiment, a sensor device 5d includes a wire-shaped pressing portion 18 having a coil shape and a sensor unit located at a distal portion of the pressing portion 18 having a coil shape. Specifically, the sensor device 5d includes a wire-shaped pressing portion having a coil shape, that is, a so-called spring-shaped pressing portion 18, and includes a plurality of sensor units 51a, 51b, and 51c that are fixed at a predetermined distance from one another at a distal portion of the pressing portion 18. Specifically, in the sensor device 5d according to this embodiment, a rear end 18a of the spring-shaped pressing portion 18 is linear, and is fixed to a shaft portion 48 of a distal end guiding portion of the stent delivery system 1f. The spring-shaped pressing portion 18 is formed in a spring shape whose distal side portion extends by a predetermined length, and the sensor units 51a, 51b, and 51c are fixed to a side surface (radially outwardly facing surface) of the spring-shaped pressing portion 18. The spring-shaped pressing portion 18 can be compressed. When the stent 3 is expanded, the pressing portion 18 is expanded, and the sensor units 51a, 51b, and 51c fixed to the side surface of the distal portion of the spring-shaped pressing portion 18 are pressed against the inner surface of the stent 3. In the sensor device 5d according to this embodiment, the sensor cables 53a, 53b, and 53c pass through the inside of the spring-shaped pressing portion 18 and enter the guide portion fixing portion 49 through an opening of the guide portion fixing portion 49. In the stent delivery system 1f according to the embodiment illustrated in FIG. 23, as in a stent delivery system 1g according to an embodiment illustrated in FIG. 24, wireless sensors 51a, 51b, and 51c having a communication function may be used as sensor units of a sensor device 5e. In this case, the sensor device 5e does not include a sensor cable.

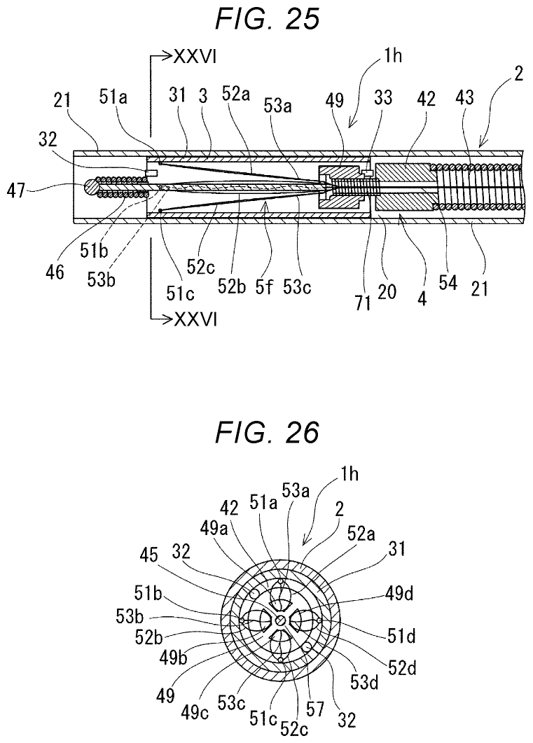

Next, a stent delivery system 1h according to an embodiment illustrated in FIG. 25 will be described. FIG. 26 is a cross-sectional view taken along a line XXVI-XXVI in FIG. 25.

In the stent delivery system 1h according to this embodiment, a sensor device 5f includes the wire-shaped pressing portions 52a, 52b, 52c, and 52d each having an elongated loop shape, and the sensor units 51a, 51b, 51c, and 51d located at the respective distal portions of the pressing portions. Specifically, in the stent delivery system 1h according to this embodiment, the sensor device 5f includes the sensor units 51a, 51b, 51c, and 51d, and the pressing portions that press the sensor units are not linear wires as in the above-described embodiment, but are formed of loop-shaped wires 52a, 52b, 52c, and 52d each having a long elliptical shape. Rear ends of the loop-shaped wires 52a, 52b, 52c, and 52d are fixed to the distal portion of the guide portion fixing portion 49. The sensor units 51a, 51b, 51c, and 51d are fixed to the respective distal portions of the pressing portions 52a, 52b, 52c, and 52d formed of the loop-shaped wires. The pressing portions 52a, 52b, 52c, and 52d formed of the loop-shaped wires can be deformed inward by pressing, and when the stent 3 is expanded, the pressing portions 52a, 52b, 52c, and 52d formed of the loop-shaped wires press the sensor units 51a, 51b, and 51c against the inner surface of the stent 3. In the sensor device 5f according to this embodiment, the sensor cables 53a, 53b, 53c, and 53d pass through the respective loop-shaped wires 52a, 52b, 52c, and 52d, and enter the stent extrusion member 4 through the opening of the guide portion fixing portion 49.

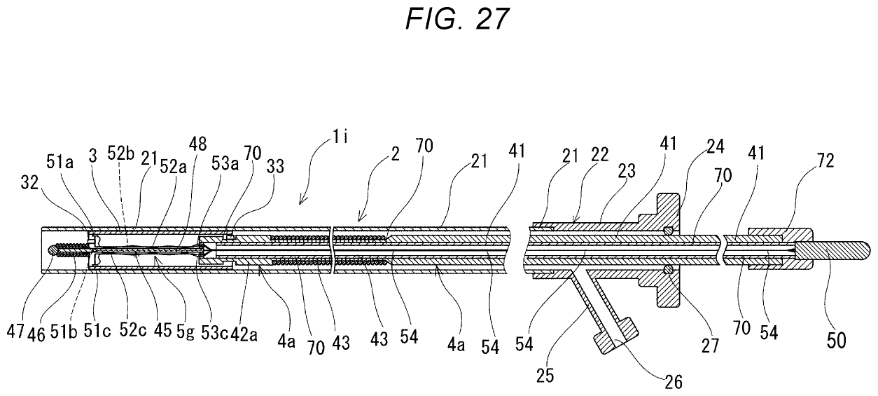

Next, a stent delivery system 1i according to an embodiment illustrated in FIG. 27 will be described. In the stent delivery system 1i according to this embodiment, a sensor device 5g includes a device tube 70 slidably inserted into or positioned in the hollow shaft portion 41 of a stent extrusion member 4a. A rear end portion of the device tube 70 protrudes from a rear end portion of the hollow shaft portion. In this embodiment, a distal end of the device tube 70 is positioned inside a distal portion of the stent extrusion member 4a, and sensor units, pressing portions, and sensor cables protrude from the distal end of the device tube 70.

In this embodiment, a distal portion of the stent pressing portion 42 of the stent extrusion member 4a is a guide portion fixing portion, and the distal end guiding portion 45 is fixed to the distal portion of the stent pressing portion 42. The rear ends of the pressing portions 52 (52a, 52b, 52c, and 52d) enter the stent pressing portion 42 from an opening portion of the stent pressing portion 42 and are bundled.

In the stent delivery system 1i according to this embodiment, the connector 50 electrically connected to the sensor cables is not fixed to the shaft hub 72, and the rear end portion of the device tube 70 is fixed to the shaft hub 72. The rear end portion of the hollow shaft portion 41 is not fixed to the shaft hub 72. Therefore, when the connector 50 is pulled rearward, the distal portion of the sensor device 5g moves rearward, and the pressing portions 52 (52a, 52b, 52c, and 52d) pass through the stent extrusion member 4a and can be accommodated in the device tube 70. When the device tube 70 is pulled rearward together with the shaft hub 72, the sensor device 5g moves rearward together with the device tube 70. If necessary, the sensor device 5g including the device tube 70 can be removed from the stent delivery system 1i by further pulling the device tube 70 rearward.

In the stent delivery system 1i according to this embodiment, the sensor cables 53 (53a, 53b, 53c, and 53d) are bundled to form a traction wire. A traction wire may be separately provided, and the sensor cables 53 (53a, 53b, 53c, and 53d) may be wound around the traction wire. When the traction wire is separately provided, the rear ends of the pressing portions 52 (52a, 52b, 52c, and 52d) are preferably fixed to a distal end of the traction wire. The sensor units 51 (51a, 51b, 51c, and 51d) may also be accommodated in the device tube 70.

In the stent delivery system 1i according to this embodiment, configurations, features and aspects other than those described above are the same as a configuration of the above-described stent delivery system 1.

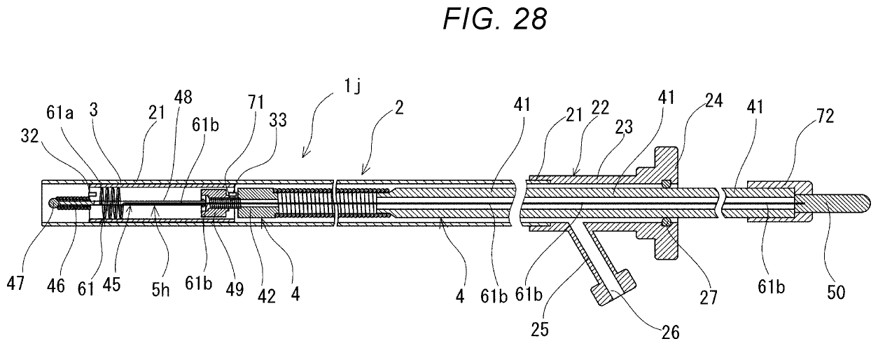

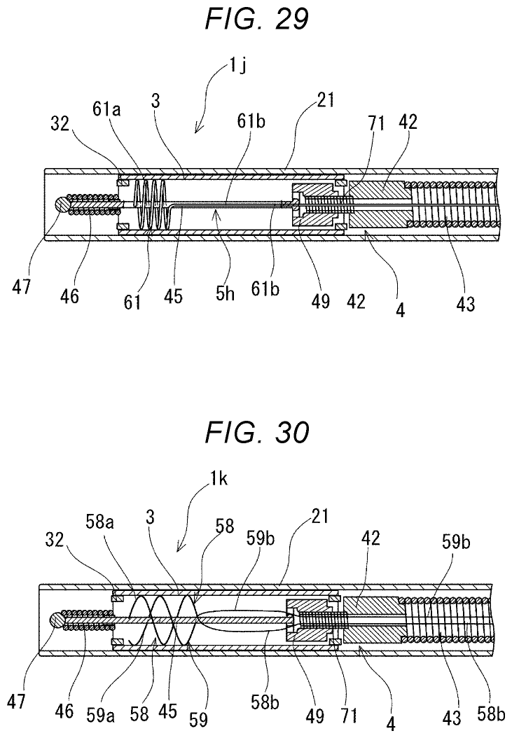

Next, a stent delivery system 1j according to an embodiment illustrated in FIG. 28 will be described. In the stent delivery system 1j according to this embodiment, a wire-shaped sensor 61 having a coil shape at a distal portion 61a is used as a sensor device 5h. As the wire-shaped sensor 61, a wire-shaped strain sensor is used. As illustrated in FIG. 29, the wire-shaped sensor 61 includes a coiled distal portion 61a, and the coiled distal portion 61a can be compressed. When the stent 3 is expanded, the coiled distal portion 61a is expanded, and a side surface (radially outwardly facing surface) of the coiled distal portion 61a is pressed against the inner surface of the stent 3. A portion behind the coiled distal portion 61a of the wire-shaped sensor 61 is a linear portion 61b, and a rear end of the linear portion 61b is electrically connected to the connector 50 through the inside of the stent extrusion member 4.

As described above, the stent delivery system disclosed here is not limited to a configuration in which the coiled distal portion 61a is formed using an elastic force of the wire-shaped sensor 61. Alternatively, a wire-shaped sensor reinforcing member in which the wire-shaped sensor 61 and a wire-shaped pressing portion disposed on a side portion thereof are integrated may be used as in the integrated members 10a, 10b, and 10c in which the pressing portions and the sensor cables illustrated in FIGS. 8 to 10 are integrated.

In the stent delivery system 1j according to this embodiment, configurations, features and aspects other than those described above are the same as the configuration of the above-described stent delivery system 1.

As the wire-shaped strain sensor, for example, a sensor having a three-layer coaxial structure including an external conductor, a piezoelectric material, and a central conductor from the outside can be used. When the strain sensor of this type is deformed from a straight state which is a standard state, a potential difference is generated between the external conductor and the central conductor according to a degree of deformation. By measuring the potential difference between the external conductor and the central conductor, the degree of deformation of the strain sensor from the standard state can be calculated. In the stent delivery system 1j according to this embodiment, one wire-shaped sensor 61 (wire-shaped strain sensor) is used, and when the coiled distal portion 61a that comes into contact with the inner surface of the stent 3 is partially deformed, a signal (data) having a potential difference different from a normal potential difference is output. For example, as illustrated in FIG. 46, when the stent 3 is expanded in a favorable state at the in-vivo indwelling portion, the coiled distal portion 61a that comes into contact with the inner surface of the stent 3 is not partially deformed and has a favorable continuous curved shape, and thus a signal of a predetermined value indicating that the stent state is favorable is output.

In this stent delivery system 1j, the arithmetic processing device 100 as illustrated in FIG. 45 is also used at the time of use, and in this case, the storage unit 107 of the arithmetic processing device 100 stores a predetermined value (normal output value or reference value) indicating that the stent state is favorable as described above. When the stent 3 is in the state illustrated in FIG. 47, the stent 3 (stent main body 31) includes the stent deformation portion 31a which is pressed by the deformation portion 16a of the cerebral artery 16 and which is curved inward. Since a part of the coiled distal portion 61a is attached to the stent deformation portion 31a, the sensor is strained at the part, and a signal (data) influenced by the strain is output. Then, the arithmetic processing device 100 compares the input signal (data) influenced by the strain with the normal output value stored in the storage unit 107. When a difference is out of a predetermined range, the arithmetic processing device 100 outputs, to the display unit, that the stent state is not favorable. Then, when it is detected that the stent state is not favorable (in the case as that in FIG. 47), after the stent delivery system 1j is removed from the living body, a balloon catheter (not illustrated) is inserted into the stent 3, and a balloon is inflated. Accordingly, the shape of the stent 3 can be improved.

When a strain is detected in a part of the distal portion of the stent during indwelling of the stent, it is possible to accommodate the stent in the sheath again, change an indwelling position, and attempt indwelling of the stent again. When such use is assumed, it is necessary to dispose the coiled distal portion 61a that comes into contact with the inner surface of the stent 3 on a part of the stent on the distal side, and for example, it is necessary to dispose the coiled distal portion 61a in a range of 50% or less of an overall length of the stent on a distal side of the stent.

A stent delivery system using a wire-shaped sensor is not limited to a stent delivery system using a single wire-shaped sensor as the above-described stent delivery system 1j.

As in a stent delivery system 1k according to an embodiment illustrated in FIG. 30, a plurality of (specifically, two) wire-shaped sensors 58 and 59 may be used. In this embodiment, as illustrated in FIG. 30, the wire-shaped sensors 58 and 59 include spiral distal portions 58a and 59a (loose coiled distal portions), and the spiral distal portions 58a and 59a can be compressed. When the stent 3 is expanded, the spiral distal portions 58a and 59a expand, and side surfaces (radially outwardly facing surfaces) of the spiral distal portions 58a and 59a are pressed against the inner surface of the stent 3. Portions behind the spiral distal portions 58a and 59a of the wire-shaped sensors 58 and 59 are linear portions 58b and 59b, and rear ends of the linear portions 58b and 59b are electrically connected to the connector 50 through the inside of the stent extrusion member 4. As the wire-shaped sensors 58 and 59, a wire-shaped sensor is used. In this embodiment, the stent delivery system is also not limited to a configuration in which coiled distal portions 58a and 59a are formed using an elastic force of the wire-shaped sensor, and a wire-shaped sensor reinforcing member in which the wire-shaped sensor and a wire-shaped pressing portion disposed on a side portion thereof are integrated may be used as in the integrated members 10a, 10b, and 10c in which the pressing portions and the sensor cables illustrated in FIGS. 8 to 10 are integrated.

In the stent delivery system 1k, the arithmetic processing device 100 as illustrated in FIG. 45 is also used at the time of use, and in this case, the storage unit 107 of the arithmetic processing device 100 stores a predetermined value (normal output value) indicating that the stent state is favorable as described above for each of the wire-shaped sensors 58 and 59. When the stent 3 is in the state illustrated in FIG. 47, the stent 3 (stent main body 31) includes the stent deformation portion 31a which is pressed by the deformation portion 16a of the cerebral artery 16 and which is curved inward. Since a part of one of the spiral distal portions 58a and 59a of the wire-shaped sensors 58 and 59 is attached to the stent deformation portion 31a, the sensor is strained at the part, and a signal (data) influenced by the strain is output.

Then, the arithmetic processing device 100 compares the input signal (data) influenced by the strain with the normal output value stored in the storage unit 107. When a difference is out of a predetermined range, the arithmetic processing device 100 outputs, to the display unit, that the stent state is not favorable. Then, when it is detected that the stent state is not favorable (in the case as that in FIG. 47), after the stent delivery system 1k is removed from the living body, a balloon catheter (not shown) is inserted into the stent 3, and a balloon is inflated. Accordingly, the shape of the stent 3 can be improved.

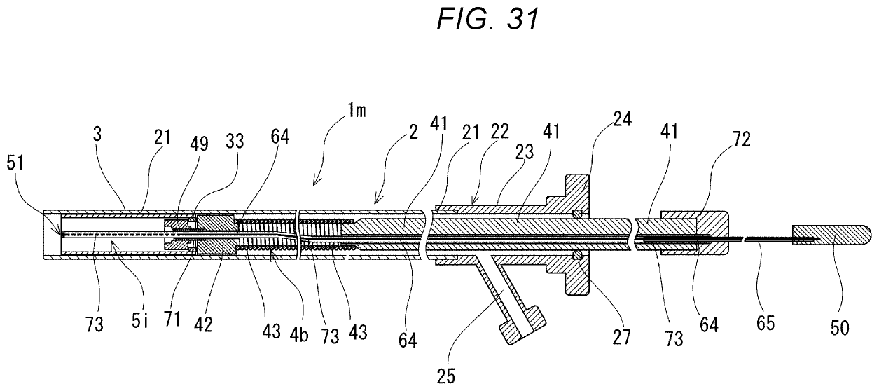

Next, a stent delivery system 1m according to an embodiment illustrated in FIG. 31 will be described.

The stent delivery system 1m according to the embodiment includes a stent extrusion member 4b and a sensor device 5i. As illustrated in FIG. 31, the stent extrusion member 4b includes a lumen opened at a distal end thereof, the sensor device 5i comprises a device tube 73 slidably accommodated in the lumen of the stent extrusion member 4b and capable of protruding from a distal end opening of the stent extrusion member 4b. The device tube 73 accommodates at least pressing portions 75a, 75b, 75c, and 75d and an operation wire 64 interlocked with rear end portions of the pressing portions in the device tube 73. The sensor units 51 (51a, 51b, 51c, and 51d) are fixed to distal portions of the pressing portions 75a, 75b, 75c, and 75d, respectively. The pressing portions 75a, 75b, 75c, and 75d are exposed from the device tube 73 by the movement of the device tube 73 toward the rear end side or the pushing of the operation wire 64, and the sensor units 51 (51a, 51b, 51c, and 51d) are brought into contact with the inner surface of the stent 3 indwelling into the living body by the pressing portions 75a, 75b, 75c, and 75d.

In this embodiment, it is preferable that the pressing portions 75a, 75b, 75c, and 75d are formed by integrating wire-shaped sensors and wire-shaped pressing portions disposed on side portions (radially outwardly facing portions) of the wire-shaped sensors as in the integrated members 10a, 10b, and 10c in which the pressing portions and the sensor cables illustrated in FIGS. 8 to 10 are integrated. The pressing portions 75a, 75b, 75c, and 75d terminate within a distal portion of the device tube 73, and behind the pressing portions 75a, 75b, 75c, and 75d, the sensor cables are bundled or twisted to form a traction wire (operation wire) 64. The traction wire 64 including the sensor cables protrudes from a rear end of the stent extrusion member 4b and is electrically connected to the connector 50.

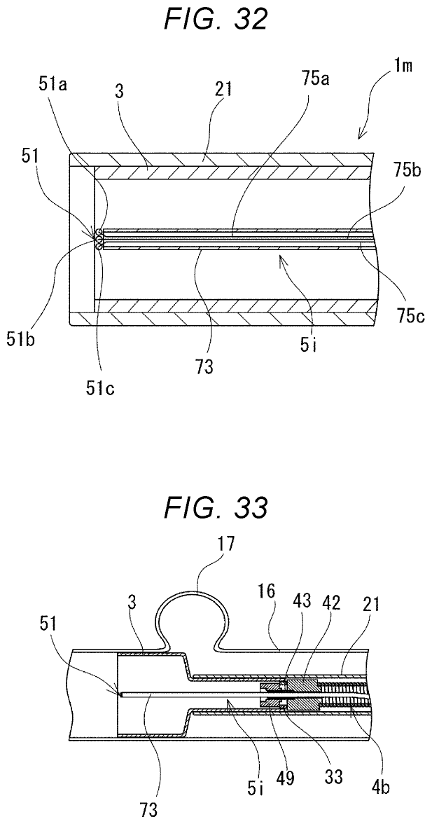

As illustrated in FIG. 31, a rear end portion of the device tube 73 of the sensor device 5i protrudes from the rear end portion of the hollow shaft portion. In this embodiment, as illustrated in FIG. 32, the distal portion of the device tube 73 protrudes from a distal end of the stent extrusion member 4b, and the distal end of the stent extrusion member 4b is located within the distal end of the stent 3. The sensor device 5i includes the sensor units 51 (51a, 51b, 51c, and 51d) and the pressing portions 75a, 75b, 75c, and 75d whose distal portions are attached to the sensor units 51 (51a, 51b, 51c, and 51d), and the pressing portions 75a, 75b, 75c, and 75d spread in a radial manner when protruding from the device tube 73.

In the stent delivery system 1m according to this embodiment, as illustrated in FIG. 31, a rear end portion of the traction wire 64 including sensor cables electrically connected to the connector 50 protrudes from the shaft hub 72. Further, a proximal portion of a protective tube 65 through which the traction wire 64 is inserted is fixed to the connector 50. A distal portion of the protective tube 65 slidably enters the shaft hub 72 and the hollow shaft portion 41. Therefore, when the connector 50 is pressed in a distal end direction, the traction wire 64 advances, and the pressing portions 75a, 75b, 75c, and 75d protrude from a distal end of the device tube 73. After the protrusion, by pulling the connector 50 in the rear end direction, the traction wire 64 is retracted, and the pressing portions 75a, 75b, 75c, and 75d can be moved back into and accommodated in the device tube 73.

The rear end portion of the device tube 73 of the sensor device 5i is fixed to the shaft hub 72. The rear end portion of the hollow shaft portion 41 is not fixed to the shaft hub 72. Therefore, when the shaft hub 72 is pulled rearward, the device tube 73 moves rearward. When the shaft hub 72 is pulled rearward in a state where the connector 50 is held, in a case where the pressing portions 75a, 75b, 75c, and 75d protrude, the pressing portions 75a, 75b, 75c, and 75d can be accommodated in the device tube 73.

By pulling the shaft hub 72, the sensor units, the pressing portions, and the traction wire can be moved rearward together with the device tube 73. If necessary, the sensor device 5i including the device tube 73 can be removed from the stent delivery system 1m by further pulling the device tube 73 rearward. The sensor units 51 (51a, 51b, 51c, and 51d) can be accommodated in the device tube 73.

It is preferable that the stent delivery system 1m according to this embodiment can detect a blood velocity which is biological-related information and check a blockage state of an aneurysm in the case where the stent indwells in the aneurysm forming portion. The blood velocity can be detected using a pressure sensor as the sensor unit 51 (51a, 51b, 51c, and 51d) and using a signal obtained therefrom. Accordingly, variations in pressure values at a plurality of sites in a lumen of a stent indwelling in a blood vessel (cerebral artery in which an aneurysm is formed) are quantified, and the presence or absence of blood flowing into the aneurysm is detected.

A step of detecting the presence or absence of blood flowing into the aneurysm using the stent delivery system 1m according to this embodiment will be described with reference to FIGS. 33 to 38.

After the stent 3 is placed in the cerebral artery 16 in which the aneurysm 17 is formed and in which the stent 3 is to indwell, specifically, after the distal end of the device tube 73 of the sensor device 5i of the stent delivery system 1m is placed in a manner of being located a predetermined distance forward (peripheral side at the predetermined distance) from the aneurysm 17 of the cerebral artery 16 having the aneurysm 17, by retracting the stent accommodation tube 21 (tube assembly 2) by a predetermined length, the stopper 33 at the rear end portion of the stent 3 is attached to and pressed by the distal end surface of the stent pressing portion 42 of the stent extrusion member 4b, and the distal side portion of the stent is discharged from the stent accommodation tube 21, resulting in a state illustrated in FIG. 33. In the state illustrated in FIG. 33, the distal side portion of the stent self-expands and is pressed into contact with the inner surface of the cerebral artery 16, and a rear end side portion of the stent is located inside the stent accommodation tube 21.

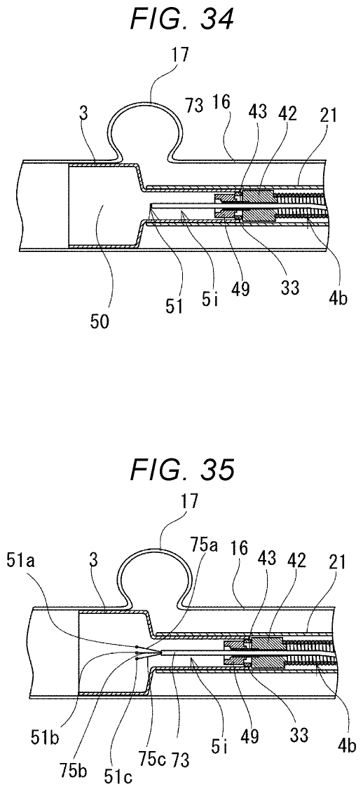

The device tube 73 and the connector 50 are pulled in the rear end direction in a state where a position of the stent accommodation tube 21 is held, and as illustrated in FIG. 34, the distal portion of the device tube 73 is located at a central portion of the stent or an aneurysm portion. Subsequently, by pressing the connector 50 in a state where a position of the device tube 73 illustrated in FIG. 34 is held, the distal portions of the pressing portions 75a, 75b, 75c, and 75d are protruded from the distal end of the device tube 73. The pressing portions 75a, 75b, 75c, and 75d each including a sensor unit at the distal portion thereof protrude. Accordingly, the pressing portions 75a, 75b, 75c, and 75d slightly spread in a radial manner and are in a state illustrated in FIG. 35. In this state, the sensor units 51 (51a, 51b, 51c, and 51d) fixed to the distal portions of the pressing portions 75a, 75b, 75c, and 75d are located near a center of the cerebral artery (blood vessel) 16 although being slightly separated from one another.

In the stent delivery system 1m according to this embodiment, the arithmetic processing device 100 as illustrated in FIG. 45 is also used. In the stent delivery system 1m according to this embodiment, in the state in FIG. 35, pressure values at four sites near the center of the cerebral artery 16 are detected using signals from the sensor units 51 (51a, 51b, 51c, and 51d) which are pressure sensors. The detected pressure value is stored in a storage unit of the arithmetic processing device 100. When the four detected pressure values are within a threshold stored in the arithmetic processing device 100, the process proceeds to the next step. When the detected pressure values vary beyond the threshold stored in the arithmetic processing device 100, by slightly pressing the connector 50 or pulling the device tube 73, the pressing portions 75a, 75b, 75c, and 75d are further exposed and the sensor units 51a, 51b, 51c, and 51d are further separated. Then, the pressure values at the four sites near the center of the cerebral artery 16 are detected again, and when the detected four pressure values are within the threshold stored in the arithmetic processing device 100, the process proceeds to the next step.

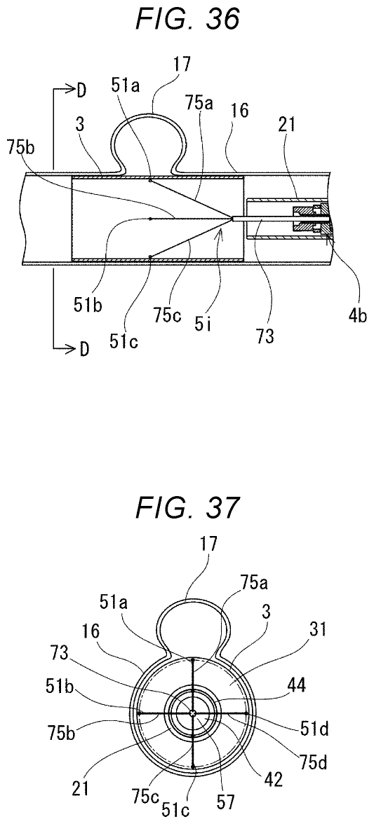

In the next step, by retracting the stent accommodation tube 21 (tube assembly 2) by a predetermined length in a state where positions of the sensor units 51 (51a, 51b, 51c, and 51d) illustrated in FIG. 35 are held, the entire stent 3 is discharged from the stent accommodation tube 21. Subsequently, the device tube 73 is pulled rearward in a state where the positions of the sensor units 51 (51a, 51b, 51c, and 51d) are held. Accordingly, all of the pressing portions 75a, 75b, 75c, and 75d protrude from the device tube 73 and spread widely in a radial manner as illustrated in FIGS. 36 and 37, and the sensor units 51 (51a, 51b, 51c, and 51d) move outward from the positions in FIG. 35 and come into contact with the inner surface of the expanded stent (stent main body 31) 3. Accordingly, the sensor units 51 (51a, 51b, 51c, and 51d) are close to the intravascular wall.

In particular, as illustrated, in this embodiment, the sensor 51a, which is one of the sensor units 51, is located near an opening portion of the aneurysm 17. Not limited to such a case, any of the sensor units is located at a site that is closer to the opening portion of aneurysm 17 than are other sensor units. In the stent delivery system 1m according to this embodiment, in states in FIGS. 36 and 37, the sensor units 51 (51a, 51b, 51c, and 51d) can be used to measure a pressure near the vascular wall. By using the signals from the sensor units 51 (51a, 51b, 51c, and 51d) which are pressure sensors, pressure values at four sites near the vascular wall of the cerebral artery 16 are detected. The detected pressure values are sent to the arithmetic processing device 100.

The arithmetic processing device 100 has a determination function of determining an embolization status of the aneurysm 17 by the stent using pressure values at four sites in a central portion of the cerebral artery 16 and the pressure values at the four sites near the vascular wall. For example, when a pressure drop exceeding a threshold is detected in one or two sensor units as compared with other sensor units in the pressure values at the four sites in the pressure near the vascular wall, it is estimated that blood flows into the aneurysm 17. When the pressure value near the vascular wall is lower than a predetermined value as compared with the pressure values detected by the same sensor unit at the central portion of the cerebral artery 16, it is estimated that blood flows into the aneurysm 17. The arithmetic processing device 100 according to this embodiment has a function of comprehensively determining the embolization status of the aneurysm 17 by the stent using the pressure values at the four sites in the central portion of the cerebral artery 16 and the pressure values at the four sites in the pressure near the vascular wall.

The arithmetic processing device 100 preferably has the following functions. When the arithmetic processing device 100 determines that blood does not flow into the aneurysm 17, the arithmetic processing device 100 displays, on the display unit 106, that the stent indwelling is favorable. When the arithmetic processing device 100 determines that blood flows into the aneurysm 17, for example, the arithmetic processing device 100 roughly estimates a flow dilation effect (inhibition of blood flow into the aneurysm from a parent artery) in the stent indwelling this time using the pressure values at the four sites in the central portion of the cerebral artery 16 and the pressure values at the four sites in the pressure near the vascular wall, and displays a result (flow dilation effect ratio) on the display unit 106. Furthermore, it is preferable that the arithmetic processing device 100 displays a precaution, a future countermeasure method, and the like on the display unit 103 based on the above result. According to the present embodiment, when it is found that a larger amount of blood flows into the aneurysm 17 than usual, it is necessary to design a treatment policy and a treatment plan in consideration of this fact. Therefore, according to the present embodiment, an appropriate treatment policy and an appropriate treatment plan according to a state of blood flow and a distribution of the blood velocity of the aneurysm 17 can be formulated.

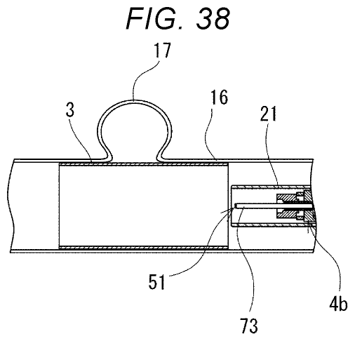

Next, after the connector 50 is pulled and the pressing portions 75a, 75b, 75c, and 75d are accommodated in the device tube 73, the device tube 73 is pulled and the distal portion in the device tube 73 is accommodated in the distal portion of the stent accommodation tube 21 (tube assembly 2), resulting in a state in FIG. 38. Then, the stent delivery system 1m is removed from the living body.

The stent delivery system 1m according to this embodiment can be used for checking the expanded state of the stent described above, in addition to the presence or absence of blood flowing into the aneurysm described above. This case will be described with reference to FIGS. 33 and 41 to 44.

First, after the stent 3 is placed in the cerebral artery 16 in which the stent 3 is to indwell, specifically, the distal end of the device tube 73 of the sensor device 5i of the stent delivery system 1m is placed in a manner of being located within the distal end of the stent 3. Subsequently, when the stent accommodation tube 21 (tube assembly 2) is retracted by a predetermined length, the stopper 33 at the rear end portion of the stent 3 is attached to and pressed by the distal end surface of the stent pressing portion 42 of the stent extrusion member 4b, and the distal side portion of the stent is discharged from the stent accommodation tube 21, resulting in the state illustrated in FIG. 33. In the state illustrated in FIG. 33, the distal side portion of the stent self-expands and is brought into contact with the inner surface of the cerebral artery 16, and the rear end side portion of the stent is located inside the stent accommodation tube 21.

By retracting the stent accommodation tube 21 (tube assembly 2) by a predetermined length in a state where the positions of the sensor units 51 (51a, 51b, 51c, and 51d) illustrated in FIG. 33 are held, the entire stent 3 is discharged from the stent accommodation tube 21. Subsequently, the device tube 73 is pulled rearward in a state where the positions of the sensor units 51 (51a, 51b, 51c, and 51d) are held. Accordingly, all of the pressing portions 75a, 75b, 75c, and 75d protrude from the device tube 73 and spread widely in a radial manner as illustrated in FIG. 41, and the sensor units 51 (51a, 51b, 51c, and 51d) come into contact with the inner surface of the expanded stent (stent main body 31) 3.

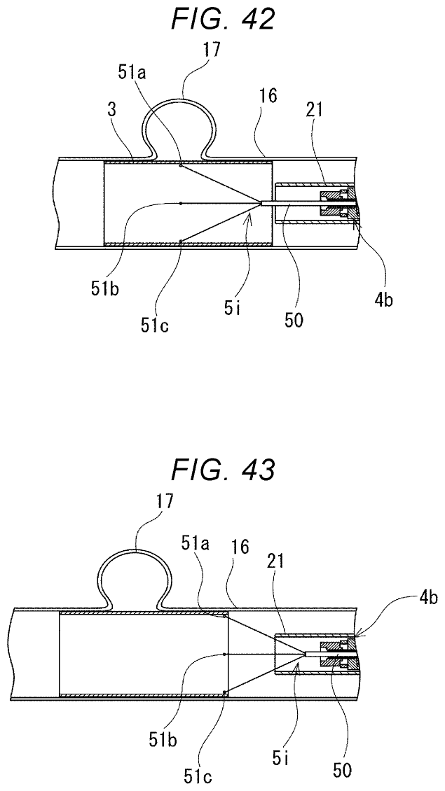

In this embodiment, since the pressing portions 75a, 75b, 75c, and 75d respectively press the sensor units 51a, 51b, 51c, and 51d against the inner surface of the stent 3 (stent main body 31) with substantially the same pressure, signals (data) caused by the output pressure are also substantially the same. In a state illustrated in FIG. 41, the stent state information (pressure values) in the stent 3 (stent main body 31) can be obtained. Further, by retracting the stent delivery system 1m from the state in FIG. 41, as illustrated in FIGS. 42 and 43, the axial positions of the sensor units 51 (51a, 51b, 51c, and 51d) in the stent 3 (stent main body 31) can be changed, and stent state information (pressure values) can be obtained at the plurality of sites.

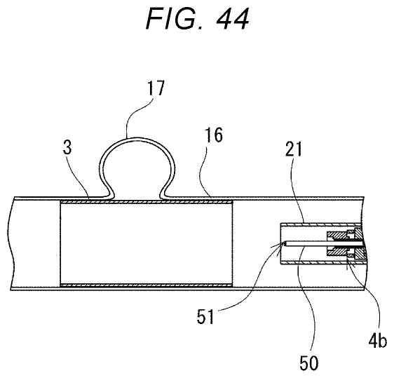

When the stent 3 is in the state as illustrated in FIG. 47, the stent 3 (stent main body 31) includes the stent deformation portion 31a which is pressed by the deformation portion 16a of the cerebral artery 16 and which is curved inward. When any one of the sensor units is attached to or comes close to the stent deformation portion 31a, the sensor is strained at this portion, and a signal (data) influenced by the strain is output. The arithmetic processing device 100 compares the input signal (data) influenced by the strain with the normal output value stored in the storage unit 107. When a difference is out of a predetermined range, the arithmetic processing device 100 outputs, to the display unit 106, that the stent state is not favorable. Then, when it is detected that the stent state is not favorable (in the case as that in FIG. 47), after the stent delivery system is removed from the living body, a balloon catheter (not illustrated) is inserted into the stent 3, and a balloon is inflated. Accordingly, the shape of the stent 3 can be improved. In this embodiment, as illustrated in FIG. 44, after the information collection is completed, the pressing portions and the device tube are accommodated in the stent accommodation tube 21, and then removed from the living body.

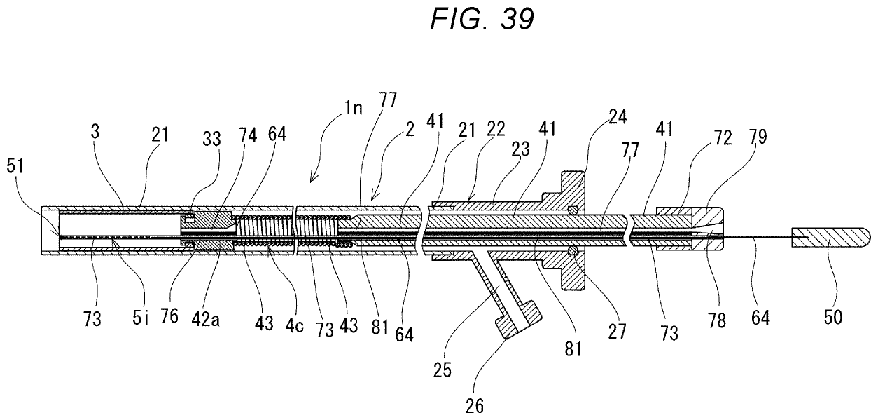

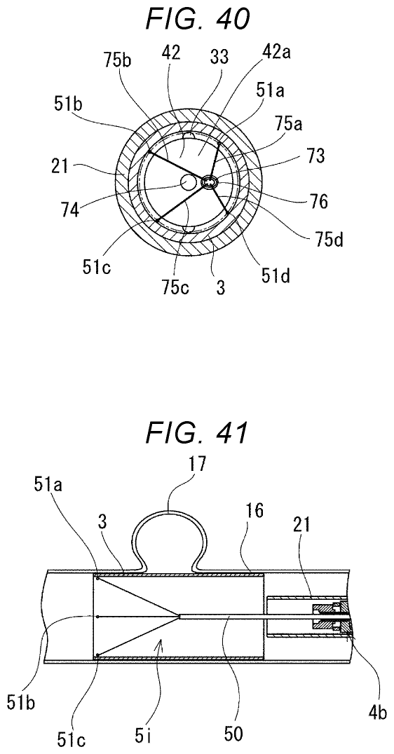

Next, a stent delivery system 1n according to an embodiment illustrated in FIGS. 39 and 40 will be described.

The stent delivery system 1n according to the embodiment includes a stent extrusion member 4c and the sensor device 5i. As illustrated in FIGS. 39 and 40, a pressing member 42a of the stent extrusion member 4c includes a tube lumen 76 that opens at a distal end thereof and that penetrates therethrough and a guide wire lumen 74. The hollow shaft portion 41 of the stent extrusion member 4c also includes a tube lumen 81 that opens at a distal end thereof and that penetrates therethrough and a guide wire lumen 77. The tube hub 79 to be described later has a guide wire insertion port 78. In the stent delivery system 1n, a guide wire (not illustrated) to be inserted from the guide wire insertion port 78 of the tube hub 79 passes through the wire lumen 77 of the hollow shaft portion 41, the elastic tubular portion 43, and the guide wire lumen 74 of the stent extrusion member 4c, and protrudes toward the distal side therefrom. Therefore, the stent delivery system 1n can be inserted into the living body and guided to a target site using a guide wire inserted through the guide wire lumen 74 at a distal portion thereof.

The sensor device 5i accommodates the device tube 73 slidably accommodated in the tube lumen 76 of the stent extrusion member 4c and capable of protruding from a distal end opening (distal end of the tube lumen 76) of the stent extrusion member (pressing member 42a) 4c, and the operation wire 64 interlocked with at least pressing portions 75a, 75b, 75c, and 75d and rear end portions of the pressing portions in the device tube 73. The sensor units 51 (51a, 51b, 51c, and 51d) are fixed to the distal portions of the pressing portions 75a, 75b, 75c, and 75d, respectively. The pressing portions 75a, 75b, 75c, and 75d are exposed from the device tube 73 by the movement of the device tube 73 toward the rear end side or the pushing of the operation wire 64, and the sensor units 51 (51a, 51b, 51c, and 51d) are brought into contact with the inner surface of the stent 3 indwelling into the living body by the pressing portions 75a, 75b, 75c, and 75d.

In this embodiment, it is preferable that the pressing portions 75a, 75b, 75c, and 75d are formed by integrating wire-shaped sensors and wire-shaped pressing portions disposed on side portions of the wire-shaped sensors as in the integrated members 10a, 10b, and 10c in which the pressing portions and the sensor cables illustrated in FIGS. 8 to 10 are integrated. The pressing portions 75a, 75b, 75c, and 75d terminate within a distal portion of the device tube 73, and behind the pressing portions 75a, 75b, 75c, and 75d, the sensor cables are bundled or twisted to form the traction wire (operation wire) 64. The traction wire 64 including the sensor cables protrudes from the rear end of the stent extrusion member 4c and is electrically connected to the connector 50.

As illustrated in FIG. 39, the rear end portion of the device tube 73 of the sensor device 5i protrudes from a rear end portion of the tube lumen 81 of the hollow shaft portion 41, and a rear end of the device tube 73 is fixed to the tube hub 79. The tube hub 79 has the guide wire insertion port 78. In this embodiment, as illustrated in FIGS. 39 and 40, the tube lumen 76 provided in the pressing member 42a of the stent extrusion member 4c is provided not at a center of the pressing member 42a but at a position on a peripheral side of (i.e., eccentric relative to or radially offset from) the pressing member 42a.

The distal portion of the device tube 73 protrudes from a distal end of the stent extrusion member 4c, and the distal end of the stent extrusion member 4c is located within the distal end of the stent 3. The sensor device 5i includes the sensor units 51 (51a, 51b, 51c, and 51d) and the pressing portions 75a, 75b, 75c, and 75d whose distal portions are attached to the sensor units 51 (51a, 51b, 51c, and 51d), and the pressing portions 75a, 75b, 75c, and 75d extend radially when protruding from the device tube 73. In particular, in this embodiment, as illustrated in FIG. 40, the tube lumen 76 is provided at a position shifted from the center of the pressing member 42a. However, the pressing portions 75a, 75b, 75c, and 75d extend radially when protruding from the device tube 73, and the sensor units 51 (51a, 51b, 51c, and 51d) are located substantially on the same circumference. In this embodiment, as illustrated in FIG. 40, the device tube 73 and the tube lumen 76 have an elliptical cross-section, and the rotary movement of the device tube 73 in the tube lumen 76 is restricted (prevented).

In the stent delivery system 1n according to this embodiment, as illustrated in FIG. 39, the hollow shaft portion 41 of the stent extrusion member 4c includes the guide wire lumen 77. The shaft hub 72 is fixed to the rear end of the hollow shaft portion 41. The rear end portion of the traction wire 64 including sensor cables protrudes from a rear end of the tube hub 79, and a rear end of the traction wire 64 is electrically connected to the connector 50. When the connector 50 is pressed in the distal end direction, the traction wire 64 advances, and the pressing portions 75a, 75b, 75c, and 75d protrude from the distal end of the device tube 73. After the protrusion, by pulling the connector 50 in the rear end direction, the traction wire 64 is retracted, and the pressing portions 75a, 75b, 75c, and 75d can be moved back into and accommodated in the device tube 73.

The rear end portion of the device tube 73 of the sensor device 5i is fixed to the tube hub 79. The rear end portion of the hollow shaft portion 41 is fixed to the shaft hub 72. However, the tube hub 79 and the shaft hub 72 are not fixed to each other. Therefore, the connector 50 and the tube hub 79 can be moved separately from the shaft hub 72. Specifically, when the tube hub 79 is pulled rearward in a state where a position of the connector 50 is held, in a case where the pressing portions 75a, 75b, 75c, and 75d protrude, the pressing portions 75a, 75b, 75c, and 75d can be accommodated in the device tube 73. Further, by pulling the device tube 73 rearward, a distal portion of the sensor device 5i can be accommodated in the stent extrusion member 4c. Further, by pulling the device tube 73 rearward, the sensor device 5i can be removed from the stent delivery system 1n. In this embodiment, by pulling the shaft hub 72, the sensor device 5i can be moved rearward together with the stent extrusion member 4c.

The stent delivery system 1 according to this embodiment can detect a blood velocity which is biological-related information, can check a blockage state of an aneurysm in the case where the stent indwells in the aneurysm forming portion, and can be used to check the expanded state of the stent.

The detailed description above describes embodiments of a stent delivery system, stent sensor device and operational methods representing examples of the new stent delivery system, stent sensor device and operational methods disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. A stent delivery system comprising:
a stent that has an inner surface and a plurality of side wall openings, that is formed in a substantially cylindrical shape and compressed in a central axis direction for insertion into a living body, and that is expandable outward for implanting into the living body;
a sheath possessing a distal portion, the stent being accommodated in the distal portion of the sheath;
a stent extrusion member that includes a distal side portion located in the distal portion of the sheath;
the sheath being movable toward a proximal end side with respect to the stent extrusion member to release the stent;
a sensor device;
the sensor device including:
a sensor unit; and
a pressing portion connected to the sensor unit to move the sensor unit into contact with the inner surface of the stent when the stent is indwelling into the living body;
the sensor unit being movable in a proximal end direction of the stent by movement of the stent extrusion member toward the proximal end side or withdrawal of the sensor device;
the sensor unit producing a signal during use of the stent delivery system that provides obtain biological-related information or stent state information in the stent; and
the sensor device being removable from the living body by removing the stent delivery system from the living body or removing the sensor device from the stent delivery system.

2. The stent delivery system according to claim 1, wherein the sensor device includes two or more of the sensor units that are separated from each other for detecting at a plurality of positions in an axial direction of the stent.

3. The stent delivery system according to claim 1, wherein the pressing portion includes a wire-shaped portion having a distal portion and the sensor unit is located at the distal portion of the wire-shaped portion.

4. The stent delivery system according to claim 1, wherein the pressing portion is a coil-shaped portion having a distal portion, the sensor unit being located at the distal portion of the coil-shaped portion.

5. The stent delivery system according to claim 3, wherein the pressing portion is comprised of a plurality of helically-shaped wires, the sensor unit being located at a distal portion of the helically-shaped wires.

6. The stent delivery system according to claim 1, wherein the pressing portion is a wire-shaped pressing portion, and a rear end portion of the wire-shaped pressing portion is fixed to the stent extrusion member.

7. The stent delivery system according to claim 1, wherein the pressing portion is a wire-shaped pressing portion, the sensor device comprises a traction wire, and a rear end portion of the wire-shaped pressing portion is interlocked with the traction wire.

8. The stent delivery system according to claim 1, wherein the stent extrusion member comprises a lumen that communicates with an open distal end of the stent extrusion member, the sensor device including a device tube accommodated in the lumen of the stent extrusion member and positionable to protrude distally beyond the open distal end of the stent extrusion member, and an operation wire interlocked with at least the pressing portion and a rear end portion of the pressing portion in the device tube, the pressing portion being relatively axially moved to a position exposed distally beyond a distal end of the device tube by rearward movement of the device tube or forward movement of the operation wire, and the sensor unit being brought into contact with the inner surface of the stent indwelling into the living body by the pressing portion when the pressing portion is exposed distally beyond the distal end of the device tube.

9. The stent delivery system according to claim 1, wherein the sensor unit comprises a wired sensor.

10. The stent delivery system according to claim 1, wherein the sensor unit comprises a wireless sensor.

11. The stent delivery system according to claim 1, wherein the sensor unit is a sensor unit that detects blood velocity, which is the biological-related information.

12. The stent delivery system according to claim 1, wherein the sensor unit is a pressure sensor.

13. The stent delivery system according to claim 1, wherein the sensor device comprises a wire-shaped strain sensor possessing the sensor unit and the wire-shaped pressing portion, and the strain sensor and the pressing portion are integrated in parallel.

14. The stent delivery system according to claim 1, further comprising an arithmetic processing device configured to calculate the biological-related information or the stent state information in the stent using the signal from the sensor unit.

15. The stent delivery system according to claim 14, wherein the sensor unit is a pressure sensor, and the arithmetic processing device outputs information on blood velocity using a difference in pressure-related signal values that are detected by the sensor unit at different positions in a radial direction of a blood vessel.

16. The stent delivery system according to claim 14, wherein the sensor unit is a blood velocity sensor, and the arithmetic processing device outputs blood vessel state information using a difference in blood velocity-related signal values that are output from three or more sensor units.

17. The stent delivery system according to claim 1, wherein the pressing portion is made of a shape memory alloy.

18. A combination of a stent sensor device and a stent;
the stent being substantially cylindrically shaped and having a plurality of side wall openings that pass through a wall of the stent from an outer surface of the stent to an inner surface of the stent, the stent being compressed radially inwardly in a central axis direction at a time of insertion of the stent sensor device and the stent into a living body, and being expandable radially outwardly at a time of indwelling the stent sensor device and the stent into the living body; and
the stent sensor device being positioned inside the stent and comprising:
at least one sensor unit positioned inside the stent, the at least one type of sensor being a wire-shaped pressure sensor, a contact force sensor, or a strain sensor;
a pressing portion connected to the sensor unit to press the sensor unit toward the inner surface of the stent; and
the sensor device being removable from a rear end side of the stent after the stent indwells into the living body.

19. The combination according to claim 18, wherein the at least one sensing unit comprises a plurality of sensing units, and the pressing portion comprises a plurality of spaced apart wire portions, each of the sensing units being connected to one of the wore portions so that the sensing units are spaced apart from one another.

20. A method comprising:
inserting a stent into a living body, the stent being substantially cylindrically shaped and having a plurality of side wall openings that pass through a wall of the stent from an outer surface of the stent to an inner surface of the stent, the stent being compressed radially inwardly during the inserting of the stent into the living body;
the inserting of the stent into the living body occurring while a sensor is positioned inside the stent;
radially outwardly expanding the stent after the stent is positioned in the living body;
moving the sensors that are positioned inside the stent toward the inner surface of the stent, the moving of the sensors toward the inner surface of the stent occurring after starting the radially outwardly expanding of the stent;
using a signal produced by the sensor to determine information about blood flowing in the living body or information about a state of the stent; and
removing the sensor from the living body while maintaining the stent in the living body.
